SEQUENCE LISTING (4 sequences)

SEQ ID NO: 1 - protein, 262 aa, synthetic construct
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 2 - protein, 262 aa, synthetic construct
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDAGSPLLCEGVFRGVTSFGLENKCGDPRGPGVYILLSKKHLNWIIMTIKGAV

SEQ ID NO: 3 - protein, 234 aa, synthetic construct
IIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 4 - protein, 234 aa, synthetic construct
IIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDAGSPLLCEGVFRGVTSFGLENKCGDPRGPGVYILLSKKHLNWIIMTIKGAV

(19) United States
(12) Patent Application Publication
(10) Pub. No.: US 2024/0100133 A1
(43) Pub. Date: Mar. 28, 2024

(54) ENZYMATICALLY INACTIVE GRANZYME A AND USES THEREFORE

(71) Applicant: Saint Louis University, St. Louis, MO (US)

(72) Inventors: Daniel F. HOFT, St. Louis, MO (US); Valerio RASI, St. Louis, MO (US)

(73) Assignee: Saint Louis University, St. Louis, MO (US)

(21) Appl. No.: 18/363,255

(22) Filed: Aug. 1, 2023

Related U.S. Application Data
(60) Provisional application No. 63/377,420, filed on Sep. 28, 2022.

(51) Int. Cl. A61K 38/48; A61P 31/06; C12N 9/64

(52) U.S. Cl. CPC A61K 38/482; A61P 31/06; C12N 9/6467; C12Y 304/21078

(57) ABSTRACT

The present disclosure is directed to recombinant mutant Granzyme A molecules that lack enzymatic activity and their use in inhibiting Mycobacterium tuberculosis and treating the related disease states.

### PRIORITY CLAIM

This application claims benefit of priority to U.S. Provisional Application Ser. No. 63/377,420, filed Sep. 28, 2022, the entire contents of which are hereby incorporated by reference.

### STATEMENT OF FEDERALLY FUNDED RESEARCH

This invention was made with government support under Award Number F30HL151136 and awarded by the National Heart, Lung, And Blood Institute and under Award Number R01AI048391 awarded by the National Institute of Allergy and Infectious Diseases. The government has certain rights in the invention.

### SEQUENCE LISTING

This application contains a Sequence Listing XML, which has been submitted electronically and is hereby incorporated by reference in its entirety. Said XML Sequence Listing, created on Jul. 30, 2023, is named USTLP0147US Sequence Listing.xml and is 6 KB in size.

### BACKGROUND

### 1. Field of the Disclosure

The present disclosure relates generally to the fields of medicine, infectious disease, and immunology. More particular, the disclosure relates to synthetic recombinant Granzyme A and its use in treating Mycobacterium tuberculosis infections.

### 2. Background

Currently one quarter of the world's population is infected with Mycobacterium tuberculosis (Mtb), the pathogen that causes tuberculosis (TB) (WHO 2020). Due to the ongoing COVID-19 pandemic, access to treatment and disease surveillance have been impacted, and recent gains against this disease have had setbacks (WHO 2021). The rise of multi-drug resistant TB has accelerated efforts to investigate novel treatments and vaccines against this disease, and new investigations are uncovering new host-pathogen interactions (Abreu et al., 2020; Nathan et al., 2008; Young et al., 2020). Mtb can efficiently infect alveolar macrophages, which in turn phagocytize and degrade this organism. However, Mtb has also developed escape mechanisms by which macrophages are no longer able to neutralize the pathogen (McClean & Tobin 2016, Wilson et al., 2019). Macrophages possess canonical surface receptors Cluster of Differentiation 14 (CD14) and Toll-like Receptors (TLR), such as TLR2 and TLR4 (Wu et al., 2019). The most studied ligand for TLR4 is lipopolysaccharide (LPS), which is a component of the outer membrane of Gram-negative bacteria (Rossol et al., 2011). LPS is known to attach to LPS-binding protein (LBP) and dock onto TLR4 and CD14 receptors for subsequent internalization and intracellular signaling through MyD88 and Trif (Tsukamoto et al., 2018; Zanoni et al., 2011). Macrophages then activate a pro-inflammatory response which leads to the production of cytokines such as TNF, IL-1β, IL-6 and interferons (Rossol et al., 2011). This inflammatory phenotype has been studied as plausible source of cytokine in bacterial sepsis models (Uranga-Murillo et al., 2021; Garzon-Tituana et al., 2021; Arias et al., 2014; Napoli et al., 2012).

Granzyme A (GzmA) is a serine protease found within the cytotoxic granules of CD8 T, NK, and γδ T cells (Arias et al., 2014). In the context of TB, GzmA has been shown to be secreted from these cells, but its protective role has not been elucidated (Uranga-Murillo et al., 2021). Each GzmA monomer has an active site with the trypsin-like catalytic triad formed by Ser195, and the two monomers are joined by a disulfide bond at Cys93 (Bell et al., 2003). GzmA is best known for its pro-apoptotic role associated with perforin, and for leading to mitochondrial disruption and cell death through its enzymatic activity (Beresford et al., 1999; Fan et al., 2003; Martinvalet et al., 2005). However, other groups have shown that physiologic concentrations of GzmA can instead induce activation of human macrophages without evidence of cell death. Moreover, GzmA can directly, and without the need of perforin, stimulate macrophages to produce inflammatory cytokines such as TNF and IL-1β (Metkar et al., 2008); synergistically potentiate LPS inflammatory response (Wensink et al., 2016); and inhibit the growth of intracellular mycobacteria within infected macrophages enzymatically independent (Rasi et al., 2021; Spencer et al., 2013). Moreover, GzmA does not act directly on the pathogen, and instead appears to potentiate activation of phagocytes (Spencer et al., 2013).

### SUMMARY

Thus, in accordance with the present disclosure, there is provided a method of treating or preventing infection of a human subject with Mycobacterium tuberculosis comprising providing to said subject an effective amount of a recombinant, homodimeric, enzymatically inactive mutant Granzyme A having a serine→alanine substitution corresponding to position 212 of SEQ ID NO: 1. The subject may have been diagnostically confirmed to have a Mycobacterium tuberculosis infection or may be at risk of contracting a Mycobacterium tuberculosis infection. The recombinant, homodimeric, enzymatically inactive mutant Granzyme A may have the sequence of SEQ ID NO: 4. The recombinant, homodimeric, enzymatically inactive mutant Granzyme A may be administered, such as intravenously, intramuscularly, subcutaneously, intranasal delivery, aerosol delivery, or inhaled. The recombinant, homodimeric, enzymatically inactive mutant Granzyme A may be provided through an expression construct, such as a viral or non-viral vector, that expresses recombinant, homodimeric, enzymatically inactive mutant Granzyme A, such as administered intravenously, intramuscularly, subcutaneously, by intranasal delivery, by aerosol delivery, or inhaled. The recombinant, homodimeric, enzymatically inactive mutant Granzyme A may be provided at about 50-200 nM. The method may further comprise administering a second Mycobacterium tuberculosis therapy, such as an antibiotic. The Mycobacterium tuberculosis may be drug resistant. The recombinant, homodimeric, enzymatically inactive mutant Granzyme A, nucleic acid or expression construct may be administered in an endotoxin free composition.

Also provided recombinant, enzymatically inactive mutant Granzyme A having a serine→alanine substitution corresponding to position 212 of SEQ ID NO: 1, or a nucleic acid or expression construct encoding the same. The recombinant, homodimeric, enzymatically inactive mutant Granzyme A may have the sequence of SEQ ID NO: 2. The mutant Granzyme A, nucleic acid or expression construct may be formulated for administration to a subject, such as a unit dose delivering 50-200 nM of mutant Granzyme A. The mutant Granzyme A, nucleic acid or expression construct may be homodimeric, lyophilized, and or is frozen.

Also provided is kit comprising the mutant Granzyme A, nucleic acid or expression construct as described herein, disposed in a receptacle, optionally further comprising instructions. Further provided is a method of producing the mutant Granzyme A as described herein comprising (a) transfecting a host cell with a nucleic acid encoding the mutant Granzyme A; (b) culturing said host cell under conditions supporting expression of the mutant Granzyme A; and (c) harvesting mutant Granzyme A from supernatants of said host cell. The method may further comprise purifying the mutant Granzyme A with column chromatography.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. The GzmA-C93S variant forms only monomers, and GzmA-monomer cannot inhibit the intracellular mycobacterial growth. (FIGS. 1A-B). GzmA-WT, but not GzmA-C93S, can form homodimers (54 kDa) under non-reducing conditions as shown by silver stain and anti-GzmA Western Blot (WB). (FIG. 1C) GzmA-C93S retains enzymatic activity as measured by the BLT cleavage assay (n=3 independent experiments, mean±SD). (FIG. 1D) GzmA-WT and GzmA-S195A inhibit intracellular mycobacterial replication, while GzmA-C93S does not (n=8 subjects from three independent experiments; mean±SEM; Wilcoxon matched-pairs signed rank test). (FIG. 1E) Mixing experiment combining GzmA-WT and GzmA-C93S shows partial inhibition of GzmA-WT activity (n=6 subjects from three independent experiments; mean and SEM; Wilcoxon matched pairs signed rank test). (***P<0.0001, **P<0.001, *P<0.01, ns=not significant).

FIGS. 2A-E. CD14 and TLR4 receptors are important for GzmA-mediated inhibition of intracellular mycobacterial growth. (FIG. 2A) Neutralization of CD14 receptor by neutralizing antibody abolishes GzmA-WT and GzmA-S195A inhibitory activities (n=8 subjects from three independent experiments; mean±SEM; Wilcoxon matched pairs signed rank test). (FIG. 2B) TLR4 was neutralized using neutralizing antibody similar to FIG. 2A. (n=8 subjects from three independent experiments; mean and SEM; Wilcoxon Matched-Pairs signed rank test). (FIG. 2C) Neutralization of TLR2 does not reverse GzmA-WT or GzmA-S195A inhibitory function (n=8 subjects from three independent experiments; mean and SEM; Wilcoxon Matched-Pairs signed rank test). (FIG. 2D) GzmA-WT and GzmA-S195A, but not GzmA-C93S, induced inhibition of mycobacteria within iMac-WT mouse cells (n=3 independent experiments; mean and SEM; student t test). (FIG. 2E) iMac-WT respond to GzmA-WT mediates intracellular mycobacterial inhibition within iMac-WT, but not CD14KO and iMac TLR4KO cells (n=3 independent experiments; mean and SEM; student t test). (***P<0.0001, **P<0.001, *P<0.01, ns=not significant).

FIGS. 3A-E. GzmA binds to mycobacteria, and its opsonization enhances monocyte inhibitory and phagocytic capacity. (FIG. 3A) Results demonstrating that GzmA-WT, GzmA-S195A, and GzmA-C93S bind to mycobacteria, while negative control protein rTS cannot (n=3 independent experiments; mean and SEM; student t test). (FIG. 3B) GzmA needs to be added to cells prior and/or during infection to induce inhibitory activity (n=8 subjects from three independent experiments; mean and SEM; Wilcoxon Matched-Pairs signed rank test). (FIG. 3C) GzmA-WT enhances phagocytosis of BCG-GFP compared to untreated cells at 2 hours post-infection (n=3 independent experiments; mean and SEM; student t test). (FIG. 3D) Rab11-FIP1 protein is upregulated in GzmA-treated and infected cells as measured by quantitative shotgun proteomics (n=4 subjects from 4 independent experiments; mean and SD). (FIG. 3E) GzmA-WT opsonizes mycobacteria, and after its excess is removed continues to inhibit the intracellular replication of the pathogen, while GzmA-C93S cannot (n=8 subjects from three independent experiments; mean and SEM). (***P<0.0001, **P<0.001, *P<0.01, ns=not significant).

FIGS. 4A-E. GzmA-WT but not GzmA-C93S interacts with CD14 and TLR4, kinetics of GzmA action, evidence of enhanced phagolysosome fusion, and translation of key findings to virulent Mtb model of infection. (FIGS. 4A-B) Different amounts of human monocyte protein lysates were incubated with α-GzmA to perform immunoprecipitation of bound proteins. These proteins were then probed for CD14 (FIG. 4A) or TLR 4 (FIG. 4B), which showed that GzmA-WT binds to CD14 and TLR4, while GzmA-C93S does not (representative figures from two independent experiments). (FIG. 4C) Human monocytes were infected with BCG-GFP and treated simultaneously (or not) with GzmA-WT and monitored over time by flow cytometry to evaluate BCG-GFP burden and GzmA expression. Results showed that the best protection was in cells that had taken up GzmA (n=4 subjects from at least two independent experiments; mean and SEM). (FIG. 4D) BCG-GFP colocalizes with LAMP1 after GzmA-WT treatment demonstrating that opsonized mycobacteria enter the phagolysosome leading to better neutralization. GzmA-C93S fails to direct mycobacteria into the phagolysosome. (Quantification of integrated density from the confocal microscopy experiments for the colocalization of mycobacteria and LAMP1 marker analyzed. Values are mean±SD of ˜40 cells per each experiment, from 2 independent experiments). (FIG. 4E) All components of GzmA effects shown with BCG infected cells were also confirmed with Mtb-infected monocytes (n=6 subjects, from 2 independent experiments; mean and SEM; Wilcoxon Matched-Pairs signed rank test). (***P<0.0001, **P<0.001, *P<0.01, ns=not significant).

FIGS. 5A-G. PDIA1 cleaves GzmA homodimers into monomers, and PDIA1 colocalizes with GzmA after treatment. (FIGS. 5A-B) Silver stained and anti-GzmA WB showing results from incubating heat-inactivated or fresh PDIA1 with GzmA-WT, and the appearance of GzmA monomer at 27 kDa. (FIG. 5C) Confocal microscopy image of human monocytes infected with mycobacteria and treated with GzmA (Red), and its colocalization (orange) with PDIA1 (green). (FIG. 5D) Deconvoluted STED images showing visual colocalization of GzmA and PDIA1 (representative figure from three independent experiments). (FIG. 5E) Percent of colocalization by quantifying Objective Pearson correlation for GzmA and ATPSH (control) or PDIA1 (data from three independent experiments; mean±SD). (FIG. 5F) Cell permeable inhibitor PACMA-31 (200 nM) enhances GzmA inhibitory activity (n=8 subjects from three independent experiments; mean and SEM; Wilcoxon Matched-Pairs signed rank test). (FIG. 5G) Similar to FIG. 5F, but cell impermeable inhibitor Rutin (50 μM) was used and showed GzmA enhancement in BCG and Mtb-infected monocytes (n=8 subjects from three independent experiments; mean and SEM; Wilcoxon Matched-Pairs signed rank test). (***P<0.0001, **P<0.001, *P<0.01, ns=not significant).

FIGS. 6A-E. (FIG. 6A) Summary figures depicting the role of GzmA in the inhibition of intracellular Mtb growth. GzmA either used as host-directed therapy (HDT) or secreted by γ9δ2 T cells opsonizes mycobacteria. Only GzmA-WT can interact with TLR4 and CD14 leading to inhibition. (FIG. 6B) GzmA-treated cells have enhanced phagocytosis and Rab11-FIP1 protein aids in the recycling of the endocytic vesicle. (FIG. 6C) Diagram showing that homodimeric GzmA feature is necessary for mediating mycobacterial growth inhibition. Monomeric GzmA cannot inhibit mycobacterial growth. (FIG. 6D) PDIA1 converts GzmA homodimer into monomer, and its inhibition potentiates GzmA inhibitory activity. (FIG. 6E) Homodimeric GzmA-treated cells show enhancement of phagolysosome fusion (figure created with BioRender.com).

FIG. 7. Sequences. Mutated and non-mutated sequences using numbering with and without propeptide sequence.

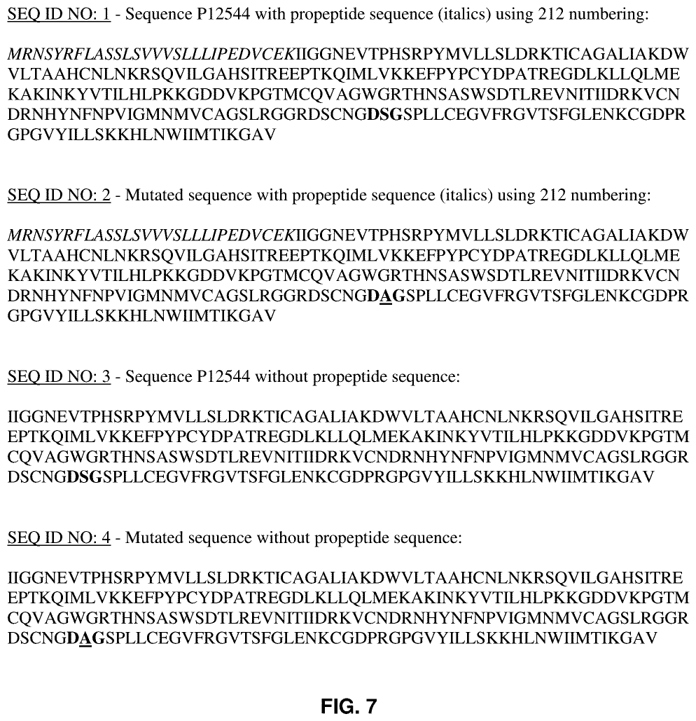

FIG. 8. Mycobacterial Growth Inhibition Assay (MGIA) results showing Disintegrations per Minute (DPM) of tritiated uridine incorporation as a marker for mycobacterial growth over 72 hours. GzmA-WT treatment inhibits the intracellular replication of mycobacteria, while GzmA-C93S does not. Mixing of GzmA-WT and GzmA-C93S shows partial inhibition (n=8 subjects from three independent experiments; mean and SEM; Wilcoxon Matched-Pairs signed rank test). (***P<0.0001, **P<0.001, *P<0.01, ns=not significant).

FIG. 9. MGIA results. Neutralizing antibodies (CD14 and TLR4) alone do not impact the bacterial burden on infected cells (n=6 subjects from three independent experiments; mean and SEM). (***P<0.0001, **P<0.001, *P<0.01, ns=not significant).

FIGS. 10A-B. (FIG. 10A) Workflow diagram for the experiment showcased in FIG. 3A. Mycobacteria expressing GFP was incubated with GzmA-WT, S195A, C93S, or rTS for 1 hour at 37° C., washed thrice with 0.1% BSA in PBS, followed by two incubations with primary and secondary antibodies before flow cytometry analysis. (FIG. 10B) Experimental setup comparing standard MGIA workflow and the GzmA-mediated opsonization of mycobacteria for experiment represented in FIG. 3E; rather than treating cells with GzmA and infecting them at the same time, mycobacteria was incubated with or without GzmA for 60 minutes at 37° C. Then, nonbounded GzmA was removed, and cells were infected.

FIGS. 11A-F. FIG. 11A) Immunoprecipitation experiment using same layout as experiment represented in FIG. 4A, but protein lysates were immunoprecipitated using isotype control antibody, and then probed for CD14, resulting in absence of detected bands (data from at least two experiments). (FIG. 11B) Same as FIG. 11A, but TLR4 was probed (data from at least two experiments). (FIGS. 11C-F) Representative confocal microscopy images for LAMP1 and BCG-GFP colocalization studies depicting BCG-GFP in green, LAMP1 in red, and nuclear stain in blue (data from two independent experiments).

FIGS. 12A-F. (FIG. 12A) Representative STED image of infected and treated cells, depicting nuclear staining in blue, GzmA in red, and PDIA1 in green (data from at least two independent experiments). (FIG. 12B) Representative confocal microscopy image showing colocalization of PDIA1 in green and GzmA in red. (FIG. 12C) Representative confocal microscopy image showing lack of colocalization of GzmA in red and ATPSH in green. (FIG. 12D) MGIA results showing PACMA-31 inhibitor alone does not impact the burden of infected cells, while it enhances inhibition when cells are treated with GzmA (n=6 subjects from three independent experiments; mean and SEM; Wilcoxon Matched-Pairs signed rank test). (FIG. 12E) MFI of PDIA1 staining following flow cytometry in human monocytes and comparison to isotype control, showing that PDIA1 is present on the cell surface. Different levels are seen in different subjects as previously reported (Stantchev et al., 2012) (n=2 subjects from at least two independent experiments; mean). (FIG. 12F) MGIA results showing that Rutin inhibitor alone does not impact mycobacterial burden within infected cells, while it enhances inhibition when cells are treated with GzmA (n=6 subjects from three independent experiments; mean and SEM; Wilcoxon Matched-Pairs signed rank test). (***P<0.0001, **P<0.001, *P<0.01, ns=not significant).

### DESCRIPTION OF ILLUSTRATIVE EMBODIMENTS

Granzyme A (GzmA) is a homodimeric serine protease found and secreted from cytotoxic T lymphocytes, NK, and γδ T cells. This enzyme is known to induce apoptosis in target cells in coordination with pore forming protein Perforin. However, this action requires intact enzymatic activity.

The inventors have previously shown that pure GzmA added extracellularly to primary human monocytes infected with Mycobacterium tuberculosis (Mtb) inhibits the intracellular replication of this pathogen, and is thus protective (Spencer, 2013). However, the enzymatic activity of GmzA has the potential for off-target adverse effects.

Mechanistic studies have now elucidated a process by which GzmA coats and opsonizes Mtb, which then interacts with monocyte surface receptors TLR4 and CD14 for more efficient degradation of the pathogen. This process requires an intact homodimeric structure but does not require an intact active site. Understanding this mechanism has allowed the inventors to mutate the GzmA sequence so that a key amino acid (serine) within the active site is converted to alanine at position 195 (tryptase sequence, or 212 in linear sequence), which mutant GzmA loses its enzymatic activity as measured by its ability to cleave peptide substrate Z-L-Lys-SBz1 hydrochloride (BLT). The inventors now demonstrate that isolated primary human monocytes treated with this enzymatically inactive variant of GzmA, the cells continued to be protected from the Mtb infection.

The inventors have established a method of efficiently producing large yields of recombinant enzymatically inactive GzmA for in vivo studies. The recombinant product is very potent at protecting against Mtb intracellular infection (similar to the native protein) and production can be scaled for clinical trials requiring GMP quality (Rasi, 2022). Thus, the inventors can produce large amounts of their synthetically created human protein that, despite its deliberate mutation, can be used to protect against Mtb infection.

These and other aspects of the disclosure are described in detail below.

### I. MYCOBACTERIUM TUBERCULOSIS

Mycobacterium tuberculosis (Mtb) is a species of pathogenic bacteria in the family Mycobacteriaceae and the causative agent of tuberculosis. First discovered in 1882, M. tuberculosis has an unusual, waxy coating on its cell surface primarily due to the presence of mycolic acid. This coating makes the cells impervious to Gram staining, and as a result, M. tuberculosis can appear weakly Gram-positive acid-fast stains such as Ziehl-Neelsen, or fluorescent stains such as auramine are used instead to identify M. tuberculosis with a microscope. The physiology of M. tuberculosis is highly aerobic and requires high levels of oxygen. Primarily a pathogen of the mammalian respiratory system, it infects the lungs. The most frequently used diagnostic methods for tuberculosis are the tuberculin skin test, acid-fast stain, culture, and polymerase chain reaction. The M. tuberculosis genome was sequenced in 1998.

M. tuberculosis was found in a complex in 2019 that has at least 9 members: M. tuberculosis sensu stricto, M. africanum, M. canetti, M. bovis, M. caprae, M. microti, M. pinnipedii, M. mungi, and M. orygis. It requires oxygen to grow, it is debated whether it produces spores, and is nonmotile. M. tuberculosis divides every 18-24 hours. This is extremely slow compared with other bacteria, which tend to have division times measured in minutes (Escherichia coli can divide roughly every 20 minutes). It is a small bacillus that can withstand weak disinfectants and can survive in a dry state for weeks. Its unusual cell wall is rich in lipids such as mycolic acid, is likely responsible for its resistance to desiccation and is a key virulence factor.

Other bacteria are commonly identified with a microscope by staining them with Gram stain. However, the mycolic acid in the cell wall of M. tuberculosis does not absorb the stain. Instead, acid-fast stains such as Ziehl-Neelsen stain, or fluorescent stains such as auramine are used. Cells are curved rod-shaped and are often seen wrapped together, due to the presence of fatty acids in the cell wall that stick together. This appearance is referred to as cording, like strands of cord that make up a rope. M. tuberculosis is characterized in tissue by caseating granulomas containing Langhans giant cells, which have a “horseshoe” pattern of nuclei.

M. tuberculosis can be grown in the laboratory. Compared to other commonly studied bacteria, M. tuberculosis has a remarkably slow growth rate, doubling roughly once per day. Commonly used media include liquids such as Middlebrook 7H9 or 7H12, egg-based solid media such as Lowenstein-Jensen, and solid agar-based such as Middlebrook 7H11 or 7H10. Visible colonies require several weeks to grow on agar plates. It is distinguished from other mycobacteria by its production of catalase and niacin. Other tests to confirm its identity include gene probes and MALDI-TOF.

Humans are the only known reservoirs of M. tuberculosis. A misconception is that M. tuberculosis can be spread by shaking hands, making contact with toilet seats, sharing food or drink, or sharing toothbrushes. However, major spread is through air droplets originating from a person who has the disease either coughing, sneezing, speaking, or singing.

When in the lungs, M. tuberculosis is phagocytosed by alveolar macrophages, but they are unable to kill and digest the bacterium. Its cell wall inhibits the fusion of the phagosome with the lysosome, which contains a host of antibacterial factors. Specifically, M. tuberculosis blocks the bridging molecule, early endosomal autoantigen 1 (EEA1); however, this blockade does not prevent fusion of vesicles filled with nutrients. In addition, production of the diterpene isotuberculosinol prevents maturation of the phagosome. The bacteria also evades macrophage-killing by neutralizing reactive nitrogen intermediates. More recently, M. tuberculosis has been shown to secrete and cover itself in 1-tuberculosinyladenosine (1-TbAd), a special nucleoside that acts as an antacid, allowing it to neutralize pH and induce swelling in lysosomes.

In M. tuberculosis infections, PPM1A levels were found to be upregulated, and this, in turn, would impact the normal apoptotic response of macrophages to clear pathogens, as PPM1A is involved in the intrinsic and extrinsic apoptotic pathways. Hence, when PPM1A levels were increased, the expression of it inhibits the two apoptotic pathways. With kinome analysis, the JNK/AP-1 signalling pathway was found to be a downstream effector that PPM1A has a part to play in, and the apoptotic pathway in macrophages are controlled in this manner. As a result of having apoptosis being suppressed, it provides M. tuberculosis with a safe replicative niche, and so the bacteria are able to maintain a latent state for a prolonged time.

Granulomas, organized aggregates of immune cells, are a hallmark feature of tuberculosis infection. Granulomas play dual roles during infection: they regulate the immune response and minimize tissue damage, but also can aid in the expansion of infection.

The ability to construct M. tuberculosis mutants and test individual gene products for specific functions has significantly advanced the understanding of its pathogenesis and virulence factors. Many secreted and exported proteins are known to be important in pathogenesis. For example, one such virulence factor is cord factor (trehalose dimycolate), which serves to increase survival within its host. Resistant strains of M. tuberculosis have developed resistance to more than one TB drug, due to mutations in their genes. In addition, pre-existing first-line TB drugs such as rifampicin and streptomycin have decreased efficiency in clearing intracellular M. tuberculosis due not being able to effectively penetrate the macrophage niche.

JNK plays a key role in the control of apoptotic pathways—intrinsic and extrinsic. In addition, it is also found to be a substrate of PPM1A activity, hence the phosphorylation of JNK would cause apoptosis to occur. Since PPM1A levels are elevated during M. tuberculosis infections, by inhibiting the PPM1A signaling pathways, it could potentially be a therapeutic method to kill M. tuberculosis-infected macrophages by restoring its normal apoptotic function in defense of pathogens. By targeting the PPM1A-JNK signaling axis pathway, then, it could eliminate M. tuberculosis-infected macrophages.

The ability to restore macrophage apoptosis to M. tuberculosis-infected ones could improve the current tuberculosis chemotherapy treatment, as TB drugs can gain better access to the bacteria in the niche, thus decreasing the treatment times for M. tuberculosis infections.

Symptoms of M. tuberculosis include coughing that lasts for more than three weeks, hemoptysis, chest pain when breathing or coughing, weight loss, fatigue, fever, night sweats, chills, and loss of appetite. M. tuberculosis also has the potential of spreading to other parts of the body. This can cause blood in urine if the kidneys are affected, and back pain if the spine is affected.

Typing of strains is useful in the investigation of tuberculosis outbreaks because it gives the investigator evidence for or against transmission from person to person. Consider the situation where person A has tuberculosis and believes he acquired it from person B. If the bacteria isolated from each person belong to different types, then transmission from B to A is definitively disproven; however, if the bacteria are the same strain, then this supports (but does not definitively prove) the hypothesis that B infected A.

Until the early 2000s, M. tuberculosis strains were typed by pulsed field gel electrophoresis. This has now been superseded by variable numbers of tandem repeats (VNTR), which is technically easier to perform and allows better discrimination between strains. This method makes use of the presence of repeated DNA sequences within the M. tuberculosis genome. Three generations of VNTR typing for M. tuberculosis are noted. The first scheme, called exact tandem repeat, used only five loci, but the resolution afforded by these five loci was not as good as PFGE. The second scheme, called mycobacterial interspersed repetitive unit, had discrimination as good as PFGE. The third generation (mycobacterial interspersed repetitive unit—2) added a further nine loci to bring the total to 24. This provides a degree of resolution greater than PFGE and is currently the standard for typing M. tuberculosis.

Antibiotic resistance in M. tuberculosis typically occurs due to either the accumulation of mutations in the genes targeted by the antibiotic or a change in titration of the drug. M. tuberculosis is considered to be multidrug-resistant (MDR TB) if it has developed drug resistance to both rifampicin and isoniazid, which are the most important antibiotics used in treatment. Additionally, extensively drug-resistant M. tuberculosis (XDR TB) is characterized by resistance to both isoniazid and rifampin, plus any fluoroquinolone and at least one of three injectable second-line drugs (i.e., amikacin, kanamycin, or capreomycin).

The genome of the H37Rv strain was published in 1998. Its size is 4 million base pairs, with 3,959 genes; 40% of these genes have had their function characterized, with possible function postulated for another 44%. Within the genome are also six pseudogenes.

The genome contains 250 genes involved in fatty acid metabolism, with 39 of these involved in the polyketide metabolism generating the waxy coat. Such large numbers of conserved genes show the evolutionary importance of the waxy coat to pathogen survival. Furthermore, experimental studies have since validated the importance of a lipid metabolism for M. tuberculosis, consisting entirely of host-derived lipids such as fats and cholesterol. Bacteria isolated from the lungs of infected mice were shown to preferentially use fatty acids over carbohydrate substrates. M. tuberculosis can also grow on the lipid cholesterol as a sole source of carbon, and genes involved in the cholesterol use pathway(s) have been validated as important during various stages of the infection lifecycle of M. tuberculosis, especially during the chronic phase of infection when other nutrients are likely not available.

About 10% of the coding capacity is taken up by the PE/PPE gene families that encode acidic, glycine-rich proteins. These proteins have a conserved N-terminal motif, deletion of which impairs growth in macrophages and granulomas. Nine noncoding sRNAs have been characterized in M. tuberculosis, with a further 56 predicted in a bioinformatics screen.

In 2013, a study on the genome of several sensitive, ultra-resistant, and multi-resistant M. tuberculosis strains was made to study antibiotic resistance mechanisms. Results reveal new relationships and drug resistance genes not previously associated and suggest some genes and intergenic regions associated with drug resistance may be involved in the resistance to more than one drug. Noteworthy is the role of the intergenic regions in the development of this resistance, and most of the genes proposed in this study to be responsible for drug resistance have an essential role in the development of M. tuberculosis.

Multidrug-resistant Tuberculosis (MDR-TB) is characterized by resistance to at least the two front-line drugs isoniazid and rifampin. MDR is associated with a relatively poor treatment success rate of 52%. Isoniazid and rifampin resistance are tightly linked, with 78% of the reported rifampin-resistant TB cases in 2019 being resistant to isoniazid as well. Rifampin-resistance is primarily due to resistance-conferring mutations in the rifampin-resistance determining region (RRDR) within the rpoB gene. The most frequently observed mutations of the codons in RRDR are 531, 526 and 516. However, alternative more elusive resistance-conferring mutations have been detected. Isoniazid function occurs through the inhibition of mycolic acid synthesis through the NADH-dependent enoyl-acyl carrier protein (ACP)-reductase. This is encoded by the inhA gene. As a result, isoniazid resistance is primarily due to mutations within inhA and the KatG gene or its promoter region—a catalase peroxidase which is required to activate Isoniazid. As MDR in M. tuberculosis becomes increasingly common, the emergence of pre-extensively drug resistant (pre-XDR) and extensively drug resistant (XDR-) TB threatens to exasperate the public health crisis. XDR-TB is characterized by resistance to both rifampin and Isoniazid, as well second-line fluoroquinolones and at least one additional front-line drug. Thus, the development of alternative therapeutic measures is of utmost priority.

An intrinsic contributor to the antibiotic resistant nature of M. tuberculosis is its unique cell wall. Saturated with long-chain fatty acids or mycolic acids, the mycobacterial cell presents a robust, relatively insoluble barrier. This has led to its synthesis being the target of many antibiotics—such as Isoniazid. However, resistance has emerged to the majority of them. A novel, promising therapeutic target is mycobacterial membrane protein large 3 (MmpL3). The mycobacterial membrane protein large (MmpL) proteins are transmembrane proteins which play a key role in the synthesis of the cell wall and the transport of the associated lipids. Of these, MmpL3 is essential; knock-out of which has been shown to be bactericidal. Due to its essential nature, MmpL3 inhibitors show promise as alternative therapeutic measures in the age of antibiotic resistance. Inhibition of MmpL3 function showed an inability to transport trehalose monomycolate—an essential cell wall lipid—across the plasma membrane. The recently reported structure of MmpL3 revealed resistance-conferring mutations associate primarily with the transmembrane domain. Although resistance to pre-clinical MmpL3 inhibitors has been detected, analysis of the widespread mutational landscape revealed a low level of environmental resistance. This suggests that MmpL3 inhibitors currently undergoing clinical trials would face little resistance if made available. Additionally, the ability of many MmpL3 inhibitors to work synergistically with other antitubercular drugs presents a ray of hope in combatting the TB crisis.

The BCG vaccine (bacille Calmette-Guerin), which was derived from M. bovis, while effective against childhood and severe forms of tuberculosis, has limited success in preventing the most common form of the disease today, adult pulmonary tuberculosis. Because of this, it is primarily used in high tuberculosis incidence regions, and is not a recommended vaccine in the United States due to the low risk of infection. To receive this vaccine in the United States, an individual is required to go through a consultation process with an expert in M. tuberculosis and is only given to those who meet the specific criteria. According to an article in 2020, a possible correlation exists between BCG vaccination and better immune response to the COVID-19.

M. tuberculosis is a clonal organism and does not exchange DNA via horizontal gene transfer. Despite an additionally slow evolution rate, the emergence and spread of antibiotic resistance in M. tuberculosis poses an increasing threat to global public health. In 2019, the WHO reported the estimated incidence of antibiotic resistant TB to be 3.4% in new cases, and 18% in previously treated cases. Geographical discrepancies exist in the incidence rates of drug-resistant TB. Countries facing the highest rates of ABR TB China, India, Russia, and South Africa. Recent trends reveal an increase in drug-resistant cases in a number of regions, with Papua New Guinea, Singapore, and Australia undergoing significant increases.

### II. GRANZYME A

Granzyme A (CTLA3, HuTPS, T-cell associated protease 1, cytotoxic T lymphocyte serine protease, TSP-1, T-cell derived serine proteinase) is an enzyme that in humans is encoded by the GZMA gene and is one of the five granzymes encoded in the human genome. This enzyme is present in cytotoxic T lymphocyte granules. Exemplary human mRNA and protein sequences are NM_006144 and NP_006135, respectively.

Cytolytic T lymphocytes (CTL) and natural killer (NK) cells share the remarkable ability to recognize, bind, and lyse specific target cells. They are thought to protect their host by lysing cells bearing on their surface “non-self” antigens, usually peptides or proteins resulting from infection by intracellular pathogens. The protein described here is a T cell- and natural killer cell-specific serine protease that may function as a common component necessary for lysis of target cells by cytotoxic T lymphocytes and natural killer cells.

This enzyme catalyzes the hydrolysis of proteins, including fibronectin, type IV collagen and nucleolin. The preferential cleavage is -Arg-, -Lys->>-Phe- in small molecule substrates.

### III. TREATMENT/PREVENTION OF M. TUBERCULOSIS INFECTION

The present disclosure provides pharmaceutical compositions comprising recombinant Granzyme A, a nucleic acid encoding the same, or a vector capable of expressing the same, and methods for generating and using the same. Such compositions comprise a prophylactically or therapeutically effective amount of recombinant Granzyme A, a nucleic acid encoding the same, or a vector capable of expressing the same, in a pharmaceutically acceptable carrier.

As discussed above, in certain embodiments, recombinant, mutant Granzyme A is administered, but in others a nucleic acid (e.g., RNA or DNA) is administered, and can be delivered in an expression cassette/vector. Expression requires that appropriate signals be provided in the construct/vector and include various regulatory elements such as enhancers/promoters from both viral and mammalian sources that drive expression of the genes of interest in cells.

There are a number of ways in which nucleic acids/expression vectors may introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. Papovaviruses (simian virus 40, bovine papilloma virus, and polyoma), adenoviruses, retroviruses, adeno-associated viruses, lentiviruses, herpesviruses, and pox viruses (vaccinia) are all viable options.

Several non-viral methods for the transfer of expression constructs into cultured mammalian cells also are contemplated by the present disclosure. These include direct microinjection, DNA-loaded liposomes, and lipofectamine-DNA complexes. Further, non-viral delivery of RNA expression constructs through modified RNAs (modRNAs), messenger RNAs (mRNAs), and micro-RNAs (miRNAs) via but not limited to endosomal, liposomal, and nanoparticles are contemplated by this present disclosure.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). Further, nucleic acid encoding the transcript related to the gene of interest may be delivered for gene augmentation via direct translation. In yet further embodiments, the nucleic acid may be stably maintained in the cell as a separate, episomal segment of DNA or RNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the disclosure, the expression construct may simply consist of naked recombinant DNA, RNA, or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA or RNA encoding a gene or transcript of interest may also be transferred in a similar manner in vivo and express the gene product.

In still another embodiment of the disclosure for transferring a naked DNA or modified RNA expression construct into cells may involve particle bombardment. This method depends on the ability to accelerate DNA or RNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA or RNA encoding a particular gene or transcript may be delivered via this method and still be incorporated by the present disclosure.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA or lipofectamine-RNA complexes.

Liposome-mediated nucleic acid delivery and expression of foreign DNA or RNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments of the disclosure, the liposome may be complexed with a hemagglutinating virus (HVJ). This has been shown to facilitate fusion with the cell membrane and promote cell entry of liposome-encapsulated nucleic acids (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA or RNA construct, it also will be desirable to include within the liposome an appropriate bacterial polymerase.

Other expression constructs which can be employed to deliver a nucleic acid encoding a particular gene or transcript into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In a specific embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, aerosol, nebulized, bronchial inhalation, intra-rectal, vaginal, topical or delivered by mechanical ventilation.

Therapeutics are envisioned where recombinant Granzyme A or vector is provided to a subject at risk of or known to be infected with M. tuberculosis. Such compositions can be formulated for parenteral administration, e.g., formulated for injection via the intradermal, intravenous, intramuscular, subcutaneous, intranasal, intraperitoneal routes, or by inhalation. Administration by intradermal and intramuscular routes are contemplated. The composition could alternatively be administered by a topical route directly to the mucosa, for example, by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In a particular embodiment, aerosol administration is contemplated. The pharmaceutical composition may be formulated as inhaled formulations, including sprays, mists, or aerosols. For inhalation formulations, the composition provided herein may be delivered via any inhalation method known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

### IV. METHODS OF PRODUCING MUTANT GRANZYME A

Recombinant mutant Granzyme A can produced as previously discussed (Rasi et al., 2022). Briefly, transient transfection of a host cell such as HEK293T cells mutant Granzyme A-encoded DNA within an expression vector. Cells are incubated with sequence-verified constructs in serum under conditions permitting vector uptake by the host cells, such as with a lipid vehicle (e.g., Lipofectamine 3000). Following incubation, mutant Granzyme A can be harvested from host cell supernatants and optionally purified using, e.g., a Ni-IMAC column, activated by enterokinase treatment, with a final MonoS column purification. Final protein can be run over an Endotrap column to remove any residual endotoxin contamination. Homodimerization may be verified by non-reducing SDS gel electrophoresis and by western blot with a GzmA antibody. Protein can be stored at −80° C. for long term storage. GzmA has been evaluated and shown to be biologically active at human physiological temperatures of 37° C. It also appears to be stable at 4° C. as frozen aliquots were frequently thawed and tested in substrate activity assays up to a week after thawing and showed that GzmA continued to retain its activity.

### V. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

GzmA purification. Recombinant GzmA was purified using an updated protocol as previously reported (Rasi et al., 2022). Briefly, transient transfection of HEK293T cells (ATCC® CRL-11268TM; ATCC, Manassas, VA) with GZMA encoded within the pHL-sec plasmid was performed. GzmA-S195A and GzmA-C93S were generated from GzmA-WT pHL-sec plasmid by site-directed mutagenesis (Genewiz; South Plainfield, NJ). Position 212 (195 using tryptase numbering) was substituted from Ala-*Ser for GzmA-S195A. For GzmA-C93S, Cys was substituted to Ala at position 105 (93 using tryptase numbering). For GzmA-WT, GzmA-S195A, and GzmA-C93S purifications, HEK293T cells were incubated at 37° C. 96 hours with sequence-verified plasmids in FBS-containing DMEM media using Lipofectamine 3000 (Cat #L3000008, Thermofisher, Waltham, MA). GzmA was then harvested from supernatants after transfection. Purification of GzmA was performed at 4° C. first using Ni-IMAC column (Cat #17531806, GE Healthcare, Chicago, IL), activated by enterokinase (Cat #SRP3032, Sigma, St. Louis, MO) treatment at room temperature (RT), and final MonoS column (Cat #17516801, GE Healthcare, Chicago, IL) purification at 4° C. All steps were completed with single-use plastic bottles and endotoxin-free reagents and the final protein was run over an Endotrap column (Lionex Gmbh, Germany) to remove any residual endotoxin contamination. Homodimerization was verified by non-reducing SDS gel electrophoresis, and proteins were verified by western blot with a GzmA antibody (1:500; R&D, Clone #356422) and further, by the lack of interaction with Granzyme K antibody (1:100; Cat #SAB2103935, Sigma, MO). Proteins were stored at −80° C. in 10 III, aliquots and thawed and diluted the day of each experiment.

BLT esterase protocol for measuring GzmA enzyme activity. BLT esterase assays and calculation of specific activity were adapted from (Rasi et al., 2021; Rasi et al., 2022). Substrate Z-L-Lys-SBz1 hydrochloride (Sigma #C3647-25MG) was added to 96 well plates with a serial dilution between 19.5-2,500 μM. Assay buffer consisted of 50 mM Tris, 154 mM NaCl, pH 7.5 in presence of 0.55 M (5,5-dithio-bis-(2-nitrobenzoic acid) (DTNB) chromophore. 120 pM of protein was added to each well and substrate hydrolysis was quantified by measuring the absorbance at 405 nm.

Human samples and monocyte isolation. Human monocytes were isolated from healthy donors as previously described (Rasi et al., 2021) and approved by the Saint Louis University IRB (protocols #26646 and #26645). Written consent from the volunteers was obtained according to the principles expressed in the Declaration of Helsinki. Briefly, frozen PBMC from leukapheresed samples were slowly resuscitated. Once thawed, monocytes were purified using EasySep Human Monocyte Isolation Kit (Cat. #19359, Stemcell), which is a CD14+ negative selection kit that also removes platelets. Flow cytometry experiments using anti-CD14 V450 (BD #561390) confirmed greater than 95% purity as expected based on the EasySep isolation kit protocol. A typical purification recovers around ˜15% CD14+ monocytes from human PBMC. For shotgun proteomic experiments, deidentified source leukocytes were purchased through Gulf Coast Regional Blood Center and human Peripheral Blood Mononuclear Cells were isolated using Ficoll Paque Plus (Cytiva #17-1440-02). Human monocytes were then purified using EasySep Human Monocyte Isolation Kit.

Preparation of Mycobacteria for infection. Connaught strain BCG or Mtb H37Rv were grown to mid-logarithmic phase in Middlebrook 7H9 media supplemented with 10% albumin, dextrose, catalase (ADC; Cat #211887 BD Diagnostics, Franklin Lakes, NJ)+0.05% Tween-80. Stocks were aliquoted in media without Tween 80 and frozen at −80° C. The concentration of the bacterial stock was determined after thawing by CFU plating performed in triplicate. Thawed aliquots were sonicated to generate single-cell suspensions before dilution and infection of monocytes. Multiplicity of Infection (MOI) was 3 for BCG and 1 for Mtb.

Mycobacterial Growth Inhibition Assay (MGIA). Primary CD14+ monocytes were plated in round-bottom 96-well plates in R+2 media (RPMI-1640+10% human HAB serum+1% L-glutamine). 1.5e5 monocytes were then infected with Connaught BCG MOI of 3 or Mtb MOI of 1 and treated with 200 nM GzmA. After overnight infection, cells were gently washed with R+2 medium three times to remove extracellular BCG or Mtb and resuspended in R+2 medium. After 72 h co-culture, cells were lysed with saponin solution in RPMI-1640, and the reaction was quenched after 2 h with 100 III, 7H9+ ADC containing 1 μCi 5,6-3H-uridine. After 72 h, plates were harvested onto glass fiber filter papers (filtermats). Filtermats received Illumina Gold F scintillation fluid and were counted using a MicroBeta2 liquid scintillation counter that measured Disintegration Per Minute (DPM). The % inhibition was calculated as: 100−100×(DPM from treated wells/DPM from untreated wells).

Expression and purification of human recombinant PDIA1. The cDNA of human PDIA1 (residues 18-479) was cloned into a pET-23 vector expression system. An N-terminal His-tag was engineered to facilitate purification. Sequence verified plasmids were transfected into BL21(DE3) cells. Cells were grown in LB medium until an OD600 of ˜0.6. Protein expression was induced with 0.5 mM IPTG. After overnight incubation at 37° C., cells were harvested by centrifugation. Soluble PDI protein was extracted by treating the cells with a nonionic detergent (B-PER, Thermo) followed by purification by affinity chromatography using a TALON resin as described before (Chinnaraj et al., 2021). Briefly, fractions eluted at 200 mM imidazole were pooled, dialyzed overnight in Tris 20 mM pH 7.4 145 mM NaCl to remove imidazole, and then loaded into size exclusion chromatography column 5200 10/300 (Cytiva) to isolate monomeric protein. Final protein concentrations were determined by measuring the absorbance at 280 nm, and using molar extinction coefficient of PDIA1 (i.e., 45755 M-lcm-1). Chemical identity was verified by mass spectrometry. Purify was >98%, as judged by SDS-PAGE.

Confocal microscopy and Stimulation Emission Depletion (STED) experiments. For confocal and STED microscopy experiments, cells were incubated on coverslips pre-treated with Cell-Tak (Corning #354240). At the time of fixation, cells were washed twice with 1×RT PBS, and then fixed in 4% paraformaldehyde for 15 min at 37° C. Slides were washed thrice for 5 min at RT with 1×PBS, and then blocked with 5% donkey serum (Cat. #017-000-121, Jackson ImmunoResearch, West Grove, PA) with 0.25% Tween-20 (Cat. #P9416, Sigma, St. Louis, MO) and True Stain Fc blocker (added only during blocking step) (Cat. #426101, Biolegend, San Diego, CA). Cells were then incubated overnight at 4° C. with primary antibody anti-GzmA (Cat. #MAB29051, R&D, Minneapolis, MN) at 12.5 μg/mL in blocking buffer, anti-PDIA1 (Cat. #MAB4236, R&D, Minneapolis, MN) at 12.5 μg/mL in blocking buffer, anti-LAMP1 at 12.5 ug/ml (Cat. #MAB4800, R&D, Minneapolis, MN), or anti-ATPSH (Cat. #ab110275, Abcam, Cambridge, UK) at 5 μg/mL in blocking buffer. To remove unbound antibody, cells were washed four times with 1×PBS at RT, and then incubated in the dark for 2 h with secondary antibody conjugated with Alexa Fluor 594 (Cat. #715-545-150, Jackson Immunoresearch, West Grove, PA) at 1:100 or Atto 647N (Cat. #ASR3248, Abcepta, San Diego, Ca) at 1:100. Cells were then washed thrice with 1×PBS at RT and incubated with 2.86 μM DAPI (Cat. #5748, Tocris, Bristol, UK) for 5 min at RT. Slides were finally mounted using ProLong Glass antifade mountant (Cat. #P36982, Life Technologies, OR, USA), cured for 48 hours, and then stored at −20° C. until visualized using a Leica SP8 TCS STED 3× (Leica Microsystems, Buffalo Grove, IL) at the Saint Louis University Research Microscopy and Histology Core. Samples were further processed by deconvolution using Huygens Professional (Scientific Volume Imaging, Netherlands).

Quantitative shotgun proteomics. 1.5e5 human monocytes were either treated or not with 200 nM GzmA, and infected or not with mycobacteria (MOI=3) overnight. After incubation, cells were then washed thrice with PBS, and the pellet lysed using lysis buffer from EasyPep Mini MS Sample Prep Kit (Cat #A40006, Thermo-Fisher, Waltham, MA) supplemented with 1:100 HALT phosphatase inhibitor (Cat #78420, Thermo-Fisher, Waltham, MA). Lysis, reduction/alkylation, protein digestion, and peptide clean-up were performed according to EasyPep recommendations. Prior to peptide cleanup, samples were labeled using TMT10plex Isobaric Label Reagent Set (Cat #90110, Thermo-Fisher, Waltham, MA) for 30 minutes, and then the reaction was quenched using EasyPep suggested reagent (5% hydroxylamine, 20% formic acid solution). After peptide cleanup, samples were dried using a vacuum centrifuge, and then resuspended in 0.1% formic acid in water for LC-MS analysis. The samples were analyzed on a Q-Exactive orbitrap MS/MS equipped with a nanospray emitter (Thermo Fisher Scientific, Waltham, MA). Nanospray parameters in positive ion mode were as follows: spray voltage, 2.0 KV; capillary temperature, 320° C.; S-lens RF level, 55. Liquid chromatography was performed using Dionex™ UltiMate™ 3000 RSLC (Thermo Scientific, MA). The pumping system comprises two different pumps: a binary rapid separation nano flow pump (NCS-3500RS UltiMate 3000) with a ternary loading pump. For the nano flow pump, the mobile phase A consisted of H₂O with 0.1% formic acid and mobile phase B consisted of 80% acetonitrile/20% H₂O/0.1% Formic Acid (FA). The loading pump mobile phase consisted of 98% H₂O/2% acetonitrile/0.1% FA. The peptides were separated by reverse phase chromatography on a C18 nano column (Acclaim PepMap C18 HPLC column 15 cm×75 μm, 2 μm particles, 100 Å pore size) with flow rate of 300 nL/min. The column temperature was maintained at 35° C. throughout the run. The samples were injected onto the column at 1% B and the peptides were separated up to 4% B for 17 mins. Then, the peptides were allowed to gradually separate up to 16% B over 100 min. Then, LC gradient went up to 25% B over 45 min, and followed by steep increase to 95% in 5 min. The gradient increased to 98% in 3 min followed by decrease to 1% B in 7 min. Mass spectra were acquired by applying an automatic data-dependent switch between an Orbitrap survey MS scan in the mass range of 350-1,500 m/z followed by collision-induced dissociation MS/MS of the top ions observed in MS scan. The automatic gain control (AGC) target was set to 3e6 with a resolution 70,000 and maximum injection time 50 ms. For MS2 analysis, the AGC target mode was set to 1e5 with a maximum injection time of 250 milliseconds and a resolution of 35,000. The isolation window was set to 1.2m/z and collision energy of 33 was used to fragment the ions. Dynamic exclusion of 40.0 s and charge exclusion of unassigned, 1.6-8, and >8 was used. The MS/MS spectra were analyzed using Sequest HT and MSPepSearch, both integrated in Proteome Discoverer 2.4.1.15 (Thermo Scientific, MA). SwissProt database for Homo sapiens (version: 2017-10-25, downloaded 07/07/2020, containing 28270 sequences) was used to search the data in Sequest HT. The NIST Human Orbitrap spectral library (NIST Human Orbitrap HCD 20160923) and ProteomeTools HCD30 PD was used to search data with MS Pepsearch. For Sequest HT database search, search parameters included trypsin, two missed cleavages, minimum peptide length of 6 and maximum peptide length of 144. Dynamic modifications of oxidation and N-terminal Acetyl were allowed. Carbamidomethyl and TMT6plex were specified as static modification. The precursor mass tolerance was set to 10 ppm and fragment mass tolerance was set to 0.02 Da for both MSPepSearch and Sequest HT. Finally, the results were subjected to statistical analysis using percolator node where the False discovery rate (FDR) was calculated using a decoy database search and only high confidence peptide identifications with FDR 0.01 were included. Identifications were accepted only for proteins with greater than 1 peptide identification and 5 PSM.

Antibody neutralization studies. Monocytes were pre-treated with anti-CD14 at 10 μg/ml (Cat. #MAB3832, R&D, Minneapolis, MN), anti-TLR4 at 10 μg/ml (Cat. #AF1478, R&D, Minneapolis, MN), anti-TLR2 at 10 μg/ml (Cat. #MAB2616, R&D, Minneapolis, MN), or isotype control at 10 μg/ml (Cat. #MAB002, R&D, Minneapolis, MN) for 30 minutes prior to GzmA treatment and infection.

iMac studies. iMac cells were a gift from Dr. Zanoni and were cultured and used as previously described (Nardo et al., 2018; Tan et al., 2015). Briefly, cells were propagated in [DMEM, 10% FBS, 5% L929-MCSF culture supernatant, 1% L-glutamine, 1% Penicillin/Streptomycin (P/S)]. On the day of infection, cells were washed twice with iMac infection media, which consists of passaging media without 5% L929-MCSF supernatant and without 1% P/S. After 1h incubation at 37° C., cells were washed once, and then were ready to be used in the same manner as human monocytes in the MGIA (see above), with the only difference that iMac infection media was used instead of R+2.

GzmA opsonization of mycobacteria. A mycobacteria BCG-GFP aliquot was thawed and sonicated thrice and washed twice in filtered PBS with 0.1% BSA. 5e6 CFU of bacteria were then incubated alone, with 200 nM of GzmA (WT, S195A, or C93S), or with 200 nM of rTS for 1 hour at 37° C. Bacteria were then washed three times with PBS solution and resuspended in anti-HIS primary antibody 1:250 (Cat. #MAB050, R&D, Minneapolis, MN) for 30 minutes at 4° C. in the dark. Then, bacteria were washed again thrice in PBS solution and resuspended in secondary antibody Anti-Mouse conjugated with Alexa Fluor 594 (Cat. #715-545-150, Jackson Immunoresearch, West Grove, PA) at 1:500 for 30 minutes at 4° C. in the dark. After incubation, bacteria were washed thrice in PBS solution, fixed with paraformaldehyde, and then processed by flow cytometry. Gating was focused on the GFP-expressing population of bacteria that was also opsonized with GzmA (anti-HIS+ for this experiment). FlowJo software was used for analysis.

Phagocytic score. Monocytes were treated or not with GzmA, after which BCG-GFP was used to infect cells for 2 hours. Cells were then washed twice with FACS buffer and processed by flow cytometry. The phagocytic score was measured as previously described (Ackerman et al., 2011).

Immunoprecipitation (IP) experiments. Monocytes were infected or not with mycobacteria and treated or not with GzmA (WT or C93S) for 16 hours. Cells were then washed thrice with PBS to remove serum proteins and lysed in IP lysis buffer (25 mM Tris pH 8.0, 150 mM NaCl, 1% NP-40, 1× protease and phosphatase inhibitors-added right before lysis). After protein quantitation by BCA, 10 or 25 μg of protein lysates per sample were incubated with pre-IP buffer-soaked Sepharose protein G beads (Cat. #101241, ThermoFisher, Waltham, MA) and 25 μg/ml of anti-GzmA antibody (Cat. #MAB29051, R&D, Minneapolis, MN) and mixed gently for 6 hours at 4° C. Beads were then washed twice with IP buffer, 2×SDS lysis buffer (non-reducing) added to each tube, vortexed, heated at 95° C. for 10 minutes, centrifuged, and collected supernatant to be used for WB. Membranes were then probed using anti-CD14 (Cat. #MAB3832, R&D, Minneapolis, MN) and anti-TLR4 (Cat. #AF1478, R&D, Minneapolis, MN).

BCG-GFP protection experiments and kinetics. Following treatment with GzmA and infection with mycobacteria, monocytes were first blocked with True Stain Fc blocker (Cat. #426101, Biolegend, San Diego, CA), and then permeabilized using permeabilization solution (Cat. #554715, BD Biosciences, San Diego, CA). Next, cells were incubated with anti-GzmA antibody conjugated with PE (Cat. #558904, BD Biosciences, San Diego, CA). A flow cytometer was then used to analyze BCG-GFP and GzmA-PE positive cells. FlowJo software was used for analysis.

PDIA1 inhibitors. PACMA-31 (Cat. #SML0838, Sigma, St. Louis, MO) was used to pretreat monocytes at 200 nM in media for 30 minutes, following GzmA treatment and infection. Similarly, Rutin (Cat. #R5143, Sigma, St. Louis, MO) was used to pretreat monocytes for 30 minutes at 50 μM in media.

Software. For generation of graphs and statistical analysis, the inventors used GraphPad Prism version 9.0.0 for Windows or Mac, GraphPad Software, San Diego, California USA, world-wide-web at graphpad.com. Images were created with BioRender.com.

### Example 2—Results

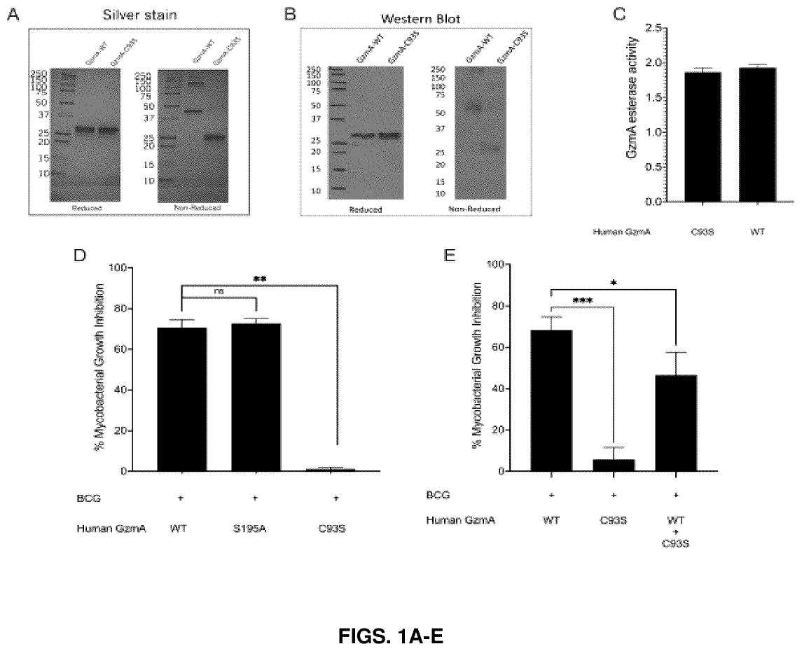

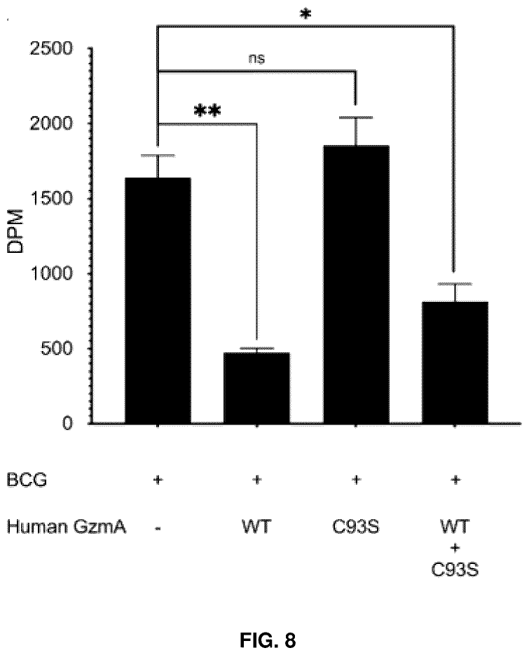

Expression and characterization of monomeric form of GzmA. The inventors recently expressed enzymatically inactive GzmA (GzmA-S195A) and demonstrated that catalytic activity is unnecessary for GzmA-mediated inhibition of intracellular mycobacterial growth in the mycobacterial growth inhibition assay (MGIA) (Rasi et al., 2021) where a strain different than Mtb was tested. To investigate whether other biochemical properties of GzmA are required for growth inhibition, the inventors substituted cysteine 93 with a serine to disrupt the intermolecular disulfide bond at this position critical for homodimeric formation (Bell et al., 2003). As demonstrated by both silver stain and western blot under non-reducing conditions, mutation of this key residue produces only the monomer form of GzmA (FIGS. 1A-B). Using the GzmA-specific substrate Z-L-Lys-SBz1 hydrochloride (BLT), the inventors demonstrated that homodimerization was not required for esterase activity as GzmA-C93S cleaved similar amounts of BLT as GzmA-WT. (FIG. 1C). Similar to previous results, enzymatically inactive GzmA-S195A inhibits the intracellular replication of mycobacteria in the MGIA (FIG. 1D). However, GzmA-C93S loses its inhibitory activity compared to both GzmA-WT and GzmA-S195A (FIG. 1D). The inventors also performed a mixing experiment in which cells were incubated with the same concentration of GzmA-WT, GzmA-C93S, or both GzmA-WT and GzmA-C93S, and the results suggest that monomeric GzmA can partially inhibit the protective effects of GzmA-WT. (FIG. 8, and FIG. 1E).

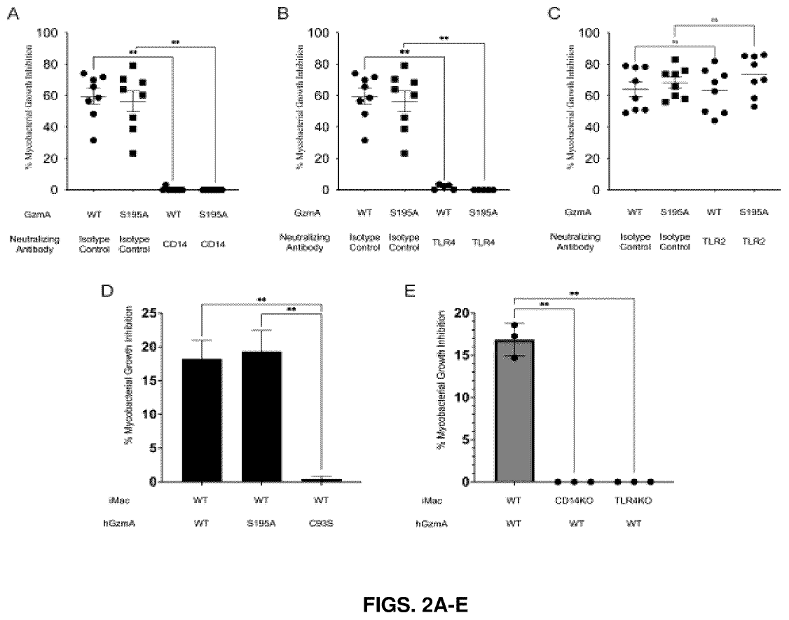

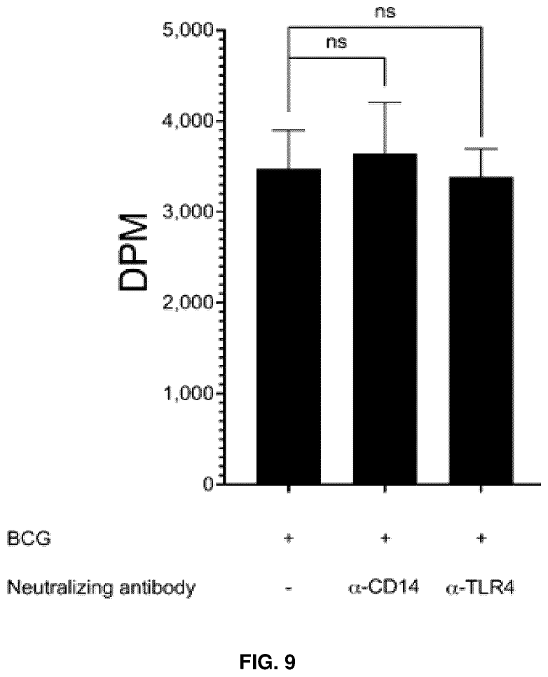

CD14 and TLR4 surface receptors are important in the GzmA-mediated inhibition in human monocytes. Multiple reports have shown that GzmA retains biological activity independent of enzymatic activity (Wensink et al., 2016; Rasi et al., 2021). Also, it has been reported that neutralizing antibody against the CD14 surface receptor reversed GzmA-induced TNF enhancement following LPS co-stimulation (Wensink et al., 2016). The inventors next determined whether CD14 neutralization would reverse GzmA-mediated mycobacterial inhibition. As observed in FIG. 2A, GzmA-WT and GzmA-S195A lost their inhibitory activity when cells were pretreated with α-CD14 (FIG. 2A) and points to GzmA-specific inhibitory axis. The CD14 receptor is known to interact with TLRs, including TLR2 and TLR4 (Wu et al., 2019; Zanoni & Granucci, 2013). Similar to the inventors' studies targeting CD14, neutralization of TLR4 reversed GzmA-mediated mycobacterial growth inhibition (FIG. 2B), while neutralization of TLR2 did not (FIG. 2C). Importantly, neutralizing antibodies against CD14 and TLR4 alone did not alter the bacterial burdens within infected monocytes (FIG. 9).

Monocytes require CD14 and TLR4 for GzmA-mediated inhibitory effects. To provide further evidence for the role of CD14 and TLR4 in the GzmA-mediated inhibition, immortalized mouse macrophage (iMac) cells were employed (Nardo et al., 2018; Tan et al., 2015). Similar to the inventors' results with human monocytes, GzmA-WT and GzmA-S195A proteins were able to modestly inhibit mycobacterial growth in iMac-WT cells. In contrast, GzmA-C93S did not (FIG. 2D). Furthermore, iMac cells that were deficient in either CD14 or TLR4 were incapable of inhibiting intracellular mycobacterial growth after treatment with GzmA-WT, confirming the results observed in the antibody neutralization studies (FIG. 2E).

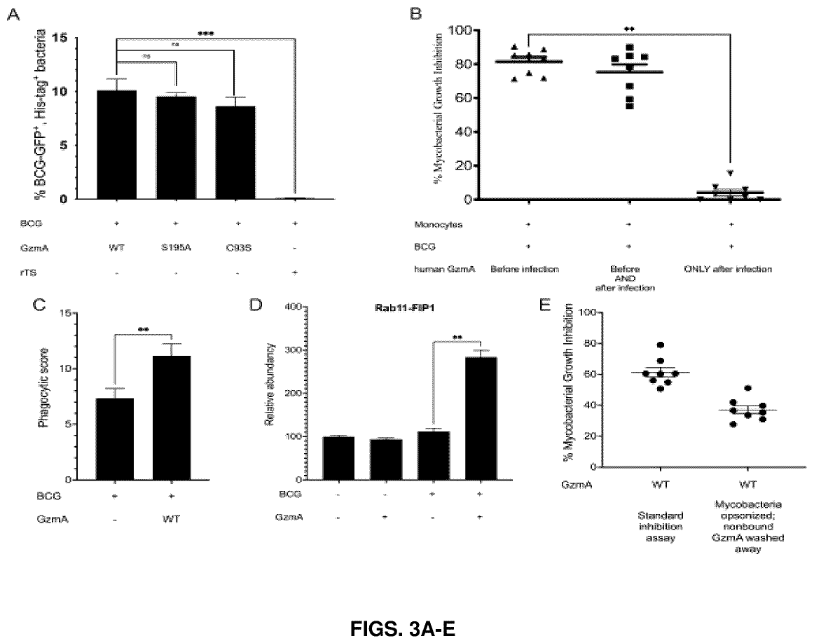

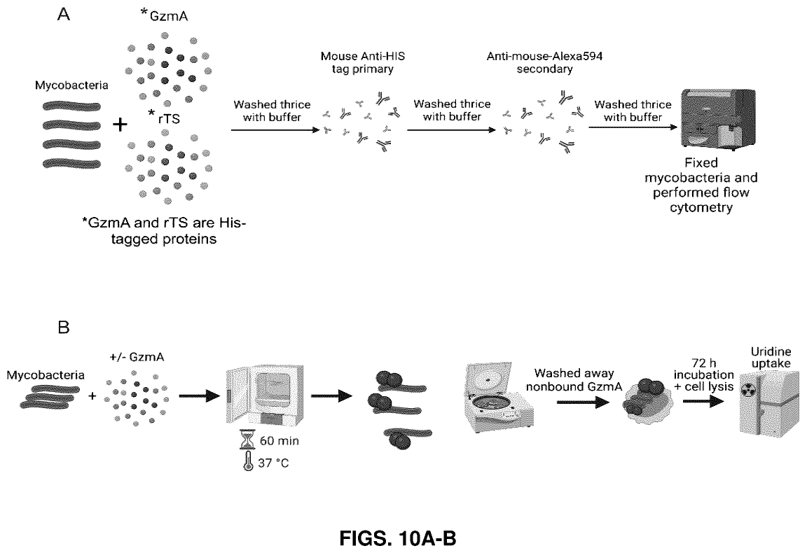

GzmA stably binds to mycobacteria. It has been shown that GzmA can stably bind to Pseudomonas aeruginosa, Neisseria meningitidis, and Escherichia coli (Wensink et al., 2016). To investigate whether GzmA similarly binds to mycobacteria, His-tagged GzmA variants were analyzed for their binding to bacteria as depicted in FIG. 10A. Both GzmA-WT and S195A bound mycobacteria (FIG. 3A), indicating that esterase activity is not required for mycobacterial binding. Surprisingly, monomeric GzmA-C93S was also able to bind mycobacteria, indicating that binding to mycobacteria is not sufficient for the inhibitory activity (FIG. 3A). As a negative control, recombinant His-tagged trans-sialidase (Hoft et al., 2007), was unable to bind mycobacteria (FIG. 3A).

GzmA acts as an opsonin enhancing mycobacterial phagocytosis. The inventors next investigated whether the addition of GzmA before, during, or after mycobacterial infection most impacted mycobacterial growth inhibition. As indicated in FIG. 3B, maximal inhibitory effects were observed when GzmA was added prior to or during mycobacterial infection; however, addition of GzmA to already infected cells did not mediate inhibition. Consistent with the opsonizing effects of complement components (Aderem & Underhill, 1999), after 2 h of infection GzmA-treated cells had a higher phagocytic score compared to untreated cells (FIG. 3C). In addition, a quantitative shotgun proteomic analysis found that Rab11-FIP1, a protein critical for optimal phagocytosis and that regulates TLR4 internalization (Damiani et al., 2004), was upregulated in the GzmA-treated and infected cells (FIG. 3D).

GzmA-opsonized mycobacteria are limited in intracellular replication capacity. Given that GzmA binds to mycobacteria, the inventors next tested whether mycobacteria incubated with GzmA-WT or PBS, then washed of nonbounded protein, would affect intracellular replication of phagocytosed mycobacteria within monocytes (schematic of experimental setup shown in FIG. 10B). Although inhibitory activity was lower compared to the inventors' standard MGIA workflow, GzmA-WT pre-opsonized mycobacteria exhibited reduced intracellular replication capacity (FIG. 3E). These data suggest that CD14 and TLR4 favor internalization of mycobacteria to then reduce mycobacterial growth.

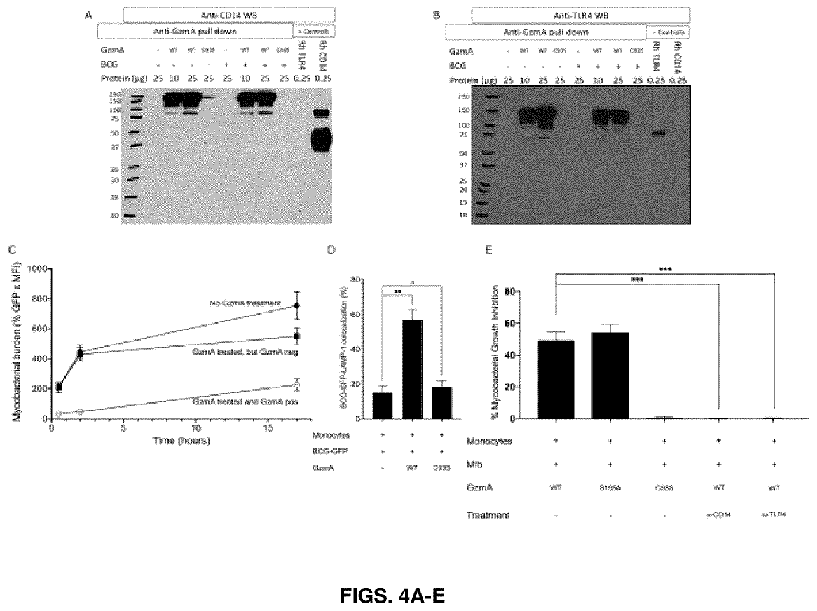

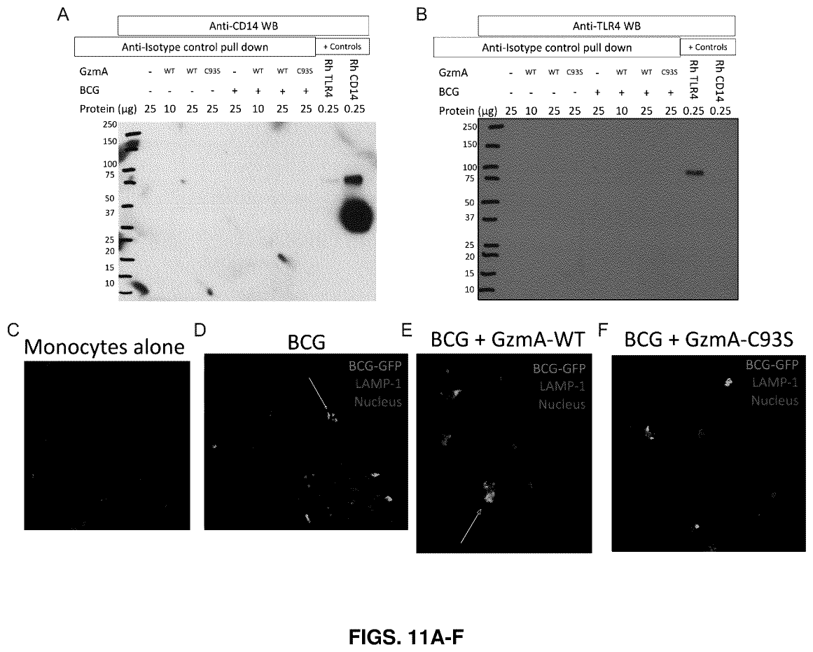

CD14 and TLR4 stably bind to GzmA. Since GzmA inhibitory activity depends on CD14 and TLR4, the inventors investigated whether GzmA could form stable interactions with these surface receptors that could be demonstrated by immunoprecipitation. Protein lysates were generated from human cells treated with either GzmA-WT or GzmA-C93S and then infected with mycobacteria. Immunoprecipitation using an anti-GzmA antibody was used to identify proteins associated with GzmA, specifically, CD14 and TLR4. The inventors found that GzmA-WT, but not monomeric GzmA-C93S, bound to CD14 (FIG. 4A). They further probed these blots and found that TLR4 also bound to GzmA-WT, but not GzmA-C93S (FIG. 4B). The interaction between GzmA and CD14/TLR4 occurred in both mycobacteria-infected and uninfected cells. Cell lysates were also immunoprecipitated with isotype control antibody, then probed for CD14 and TLR4 WB as controls (FIGS. 11A-B).

Monocytes internalizing GzmA are better protected from infection. To further study the GzmA inhibitory effects, cells were infected with GFP-expressing mycobacteria and then analyzed by flow cytometry. Cells without GzmA treatment had a rapid uptake of mycobacteria followed by an increase in growth over 18 h (FIG. 4C). Cells that were treated with GzmA but did not have sufficient internalization of protein (GzmA-), exhibited a similar rapid uptake of mycobacteria, but had reduced mycobacterial growth over time. Furthermore, when GzmA could be detected in treated monocytes, these cells exhibited the least uptake and persistence of bacteria. Overall, there was a 78% reduction in mycobacterial growth AUC for cells treated and staining positive for GzmA.

GzmA-opsonization of mycobacteria results in enhanced phagolysosome fusion. To understand the downstream effects of GzmA-opsonization on phagocytosed mycobacteria, monocytes were infected with BCG expressing GFP, GzmA-treated, and stained with LAMP 1. LAMP1 is a lysosomal protein used as a marker of late phagosome-lysosome fusion to study colocalization of mycobacteria within the phagolysosome (Park et al., 2019). The inventors' studies showed that there is a greater degree of colocalization between LAMP1 and BCG-GFP after GzmA-WT treatment compared with untreated controls or with GzmA-C93S treatment (FIG. 4D), and representative figures in (FIGS. 11C-F).

GzmA treatment has identical effects on Mtb-infected human monocytes. Although BCG closely mimics the infection kinetics and can elicit a similarly strong cellular response as Mtb, it was important to verify that the inventors' studies could be generalizable to fully virulent Mtb. Thus, human monocytes were infected with Mtb and treated with GzmA-WT, S195A, and C93S. Similar to the effects of GzmA on intracellular BCG, both enzymatically active and inactive homodimeric GzmA inhibited Mtb, whereas monomeric GzmA did not (FIG. 4E). Neutralization of CD14 and TLR4 also reversed GzmA-mediated mycobacterial growth inhibition (FIG. 4E). Thus, these studies are generalizable to virulent Mtb strengthening the biological significance of the inventors' findings.

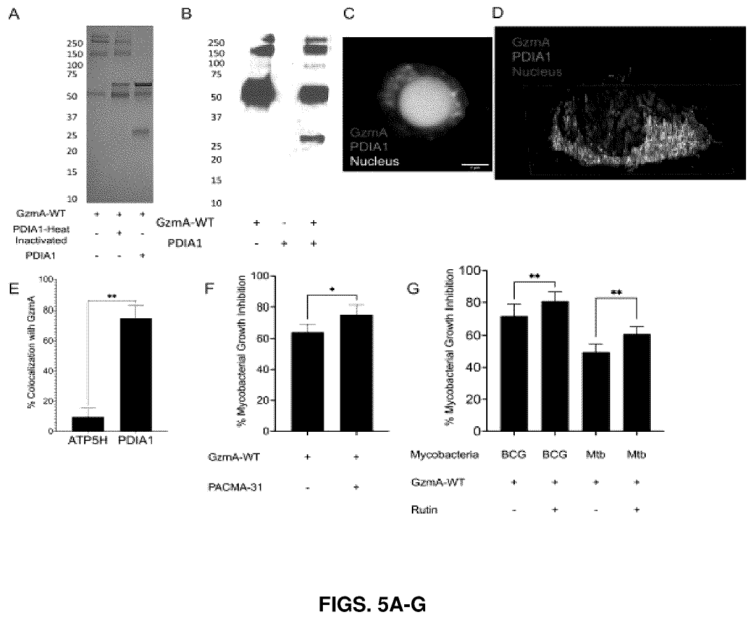

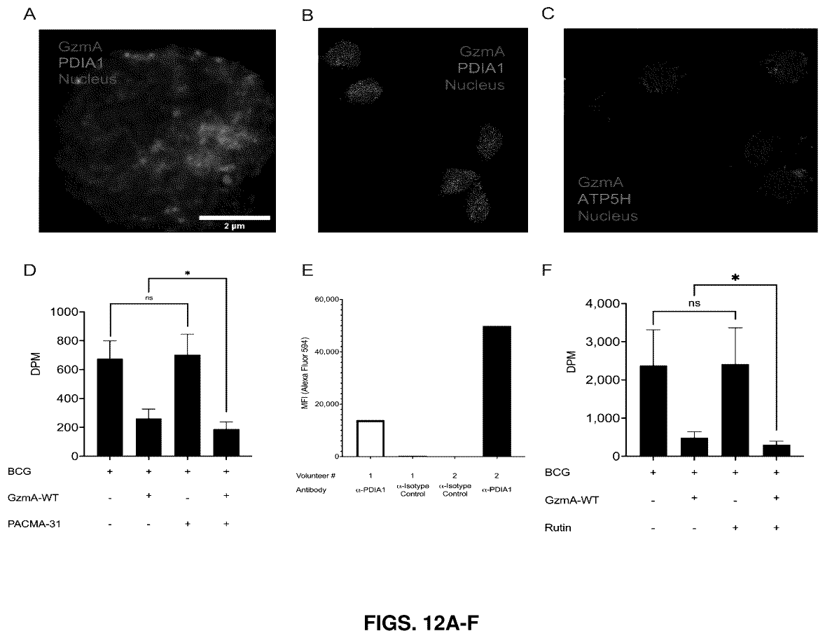

Protein Disulfide Isomerase A1 (PDIA1) cleaves and colocalizes with GzmA. Previous study showed that Protein Disulfide Isomerase A1 (PDIA1) is upregulated in human monocytes following mycobacterial infection (31% compared to control) and significantly more in cells infected and treated with GzmA (90% compared to control) (Rasi et al., 2021). Based on previous reports showing that PDIA1 can cleave disulfide bonds and is found on the surface of human cells (Stantchev et al., 2012; Wan et al., 2012) and in human plasma (Oliveira et al., 2019), and that GzmA-C93S cannot inhibit mycobacteria, the inventors next investigated the effects of PDIA1 on GzmA. First, they incubated GzmA-WT with PDIA1 and observed the monomeric band at 27 KDa, which was not present when heat-inactivated PDIA1 was used (FIG. 5A). To verify this was indeed the GzmA monomer, blots were probed with an antibody against GzmA. As demonstrated in FIG. 5B, the antibody recognized the 27 KDa protein demonstrating that this band is the GzmA monomer. To assess co-localization, cells were analyzed by immunocytochemistry, and as indicated in FIG. 5C, there was significant overlap between GzmA and PDIA1 (orange regions). Since the resolution of traditional immunocytochemistry is limited (˜200 nm), Stimulated Emission Depletion (STED) microscopy was employed to better visualize this co-localization (˜50 nm resolution) (FIG. 12A) (34). Using software deconvolution, the inventors obtained images that showed the overlap of GzmA and PDIA1 as seen in orange in FIG. 5D. To quantify the overlap, multiple STED images were analyzed which indicated a high degree of co-localization (75%) between GzmA and PDIA1, while a low degree was found with ATP5H, which is a mitochondrial protein previously found to be upregulated by GzmA, but of unknown importance (Rasi et al., 2021) (FIG. 5E). Representative images are shown in FIGS. 12B and 12C.

PDIA1 inhibitors enhance GzmA-mediated inhibition of intracellular mycobacteria. Since PDIA1 cleaves GzmA-WT into a monomer that lacks inhibitory activity, the inventors tested whether PDIA1-specific inhibitors would enhance GzmA-WT inhibitory activity within mycobacteria-infected cells. The cell permeable PDIA1 inhibitor PACMA-31 did not influence the intracellular bacterial burden when given alone (FIG. 12D) but combined with GzmA-WT enhanced mycobacterial inhibitory activity (FIG. 5F). Reports have shown that in addition to ER localization, cells can express PDIA1 on their surface (Stantchev et al., 2012; Wan et al., 2012). Flow cytometry was used to confirm that human monocytes express PDIA1 protein on the cell surface compared to isotype control (FIG. 12E). To further identify the location of PDIA1 effects, the inventors used the cell impermeable PDIA1 inhibitor Rutin and found that it also enhanced GzmA-mediated inhibition, presumably by limiting the proposed turnover of GzmA-WT into monomer (FIG. 5G and FIG. 12F). The enhancement of GzmA-mediated mycobacterial inhibition was seen with both BCG and Mtb infections. Thus, PDIA1 enhancing effects on GzmA-mediated mycobacterial growth inhibition appear to occur at the monocyte cell surface.

### Example 3—Discussion

The activation status of monocytes early during tuberculosis infection is a key determinant for disease susceptibility (Cohen et al., 2018), and the understanding of the human immune landscape may influence novel host-directed therapies (Bastos et al., 2017). The discovery that γ9δ2 T cells present at lung and mucosal surfaces exhibit potent inhibitory effects on Mtb infected macrophages points to a potential therapeutic window that deserves attention (Abate et al., 2016; Hoft et al., 1998; Spencer et al., 2008; Worku & Hoft, 2003; Xia et al., 2016). Moreover, GzmA is key for γ9δ2 T cell mediated Mtb inhibitory effects (Spencer et al., 2013).

Since GzmA's enzymatic activity is not necessary for the inhibition of intracellular mycobacteria, a structural biology approach was taken to investigate homodimerization as key determinant for protection. Here, the inventors demonstrate that homodimerization is required for the inhibition of intracellular pathogen growth. To the best of the inventors' knowledge, this is the first time that GzmA's monomeric nature was interrogated in biological assays. Moreover, proteomic studies found upregulation of PDIA1 expression following mycobacterial infection (22). Given PDIA1 disulfide bond breaking capacity, it led us to investigate the potential role for PDIA1 in converting GzmA homodimers into monomers. As shown here, PDIA1 converts GzmA homodimer into a monomer (FIGS. 5A-B) and PDIA1 inhibitors (5F and 5G) enhance GzmA inhibitory effects on mycobacteria. Clinical trials testing PDIA1 as pharmaceutical intervention in treating thrombosis and diabetes are ongoing (Gaspar & Gibbins 2021). Given PDIA1's presence in plasma of human samples (Oliveira et al., 2019) and on the cell surface of cells (Stantchev et al., 2012; Wan et al., 2012), the inventors interrogated and found PDIA1 to also be expressed on the cell surface of human monocytes. This suggests that the use of PDIA1 inhibitors may be an attractive avenue to enhance GzmA effects without other intracellular off target effect. Moreover, there is evidence of higher thrombotic events in active TB patients compared to the general population (Danwang et al., 2021). Thus, use of PDIA1 inhibitors could function as both anti-thrombotic agents as well as host-directed therapy to enhance immune responses against Mtb. Furthermore, studies have shown higher GzmA levels in serum of septic patients, with subsequent increase in pro-inflammatory cytokines (Garzon-Tituana et al., 2021). In sepsis, a different approach where the turnover to a less biologically active GzmA may be achieved by potentiating PDIA1. Future studies will investigate whether monomeric GzmA shows synergistic and pro-inflammatory effects with LPS in human monocytes.

The inventors found both CD14 and TLR4 are important for the inhibition of mycobacterial growth (FIGS. 2A-B). TLR4 has been shown to enhance CD14- and LBP-triggered signaling in monocytes, leading to the production of IL-1β, IL-6, IL-8, and TNF (Rajaiah et al., 2015). iMac studies using KO cell lines confirmed the importance of CD14 and TLR4 for GzmA-mediated inhibition (FIGS. 2D-E). The more modest GzmA-mediated inhibition observed with iMac cells could be explained by the use of human GzmA with mouse cells.

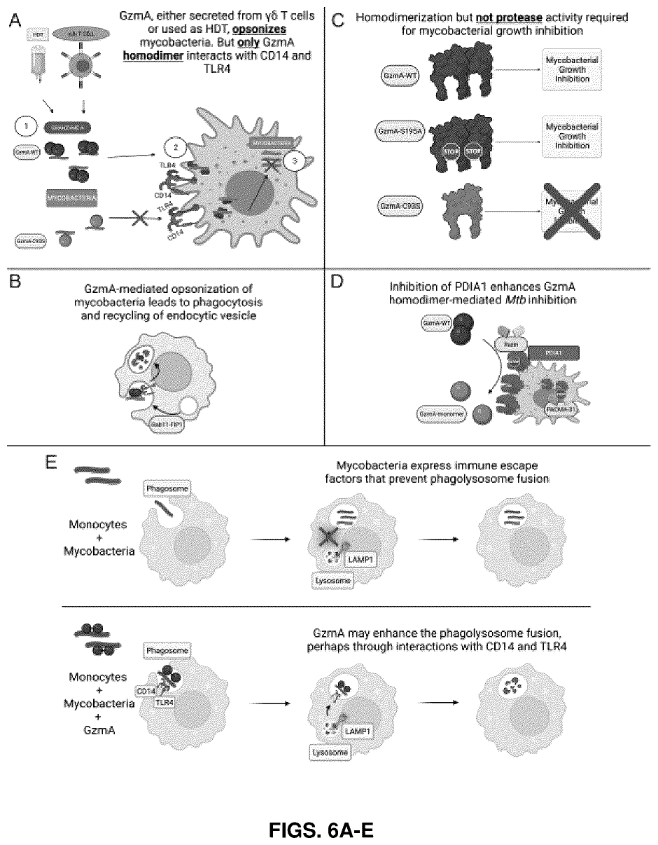

Based on previous reports, it appears that GzmA strongly binds to some Gram-negative bacteria (Wensink et al., 2016). Mycobacteria do not efficiently take up the Gram stain due to the thick layer of mycolic acid (Maitra et al., 2019). The inventors tested whether GzmA stably binds to mycobacteria and found that both homodimer and monomer versions bind to the bacteria (FIG. 3B). Thus, the GzmA protein structure can bind to components of the bacteria (likely the outer mycolic acid layer), and future studies will investigate which cell wall components are necessary for this interaction. Taken together, these data demonstrates that GzmA homodimer opsonizes mycobacteria for phagocytosis in parallel with the upregulation of Rab11-FIP1 (FIGS. 6A-E). To the best of the inventors' knowledge, this is the first report that evidences a GzmA-mediated opsonization effect against a pathogen, and future research will investigate the implications against other microbes. Recent data indicates that Rab11-FIP1 aids in the recycling of endocytic vesicles during an incoming phagocytic event (Lindsay et al., 2002). Previous studies have shown that phagocytosis is severely impaired in Rab11-FIP1 KO cells (Damiani et al., 2004). Thus, these data suggest that GzmA-tagged mycobacteria signals through TLR4 and CD14, and Rab11-FIP1 increases the phagocytic process, which enhances the degradation of the mycobacteria within the phagolysosome Mtb has developed many immune evasion mechanisms, some of which rely on inhibiting phagolysosome fusion (Hmama et al., 2015). The higher degree of colocalization of LAMP1 and mycobacteria highlights the restoration of phagolysosome fusion induced by GzmA (FIG. 4D). Previous studies showed that the ER stress response and ATP producing proteins are important components of the GzmA-mediated intracellular pathogen degradation process, and future studies will investigate whether TLR4 and CD14 signaling triggers the antimicrobial properties. A recent study found that Rv3463 interacts through the TLR4 pathway, which leads to the enhancement of phagolysosomal fusion and pathogen clearance (Park et al., 2019). Another study showed that GzmA-triggered inflammatory responses were abrogated in TLR4KO macrophages or WT cells pretreated with TLR4 inhibitors (Uranga-Murillo et al., 2021). Recent studies have also shown the role of TLR4 in aiding the phagocytosis of pathogens and its redirection in TLR4-containing endosomes (Taguchi & Mukai, 2019). These results extend these earlier observations indicating that GzmA opsonizes mycobacteria and redirects the pathogen into a direct phagolysosomal maturation dependent on TLR4 and CD14 that leads to efficient neutralization of the pathogen.

These studies did not investigate the in vivo effects of GzmA in humans because of the need to use GMP material for clinical studies. Separately, the inventors have not investigated the effects of GzmA in vivo in mice because of the known differences between human and murine GzmA, both at the sequence and functional levels. Furthermore, there are functional redundancies between different mouse granzymes given the larger family of proteins found in mice (five in humans and eight in mice), different substrate specificities between mouse and human GzmA, mouse GzmA is cytotoxic compared to human GzmA, and lack of increased susceptibility in GZMA−/− mice challenged with Mtb (Kaiserman et al., 2006; Uranga et al., 2016). Future research will investigate GzmA in vivo treatments in Mtb infected NHP, humanized mice, and in humans. However, the inventors' key findings were reproducible across models of attenuated BCG and Mtb infection of monocytes.

In conclusion, GzmA homodimer opsonizes mycobacteria to form strong interactions with CD14 and TLR4 that leads to phagocytosis. This event likely enables cells to direct the incoming infection for better degradation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
Abreu et al., “Host-Pathogen Interaction as a Novel Target for Host-Directed Therapies in Tuberculosis.” Front Immunol 11, 1553 (2020).
Abate et al., “Mycobacterium-Specific 79A2 T Cells Mediate Both Pathogen-Inhibitory and CD40 Ligand-Dependent Antigen Presentation Effects Important for Tuberculosis Immunity.” Infect Immun 84, 580-589 (2016).
Ackerman et al., “A robust, high-throughput assay to determine the phagocytic activity of clinical antibody samples.” J Immunol Methods 366, 8-19 (2011).
Aderem & Underhill, “Mechanisms of phagocytosis in macrophages.”Annu Rev Immunol 17, 593-623 (1999).
Arias et al., “Elucidating sources and roles of granzymes A and B during bacterial infection and sepsis.” Cell Rep 8, 420-429 (2014).
Bastos et al., “The Troika Host-Pathogen-Extrinsic Factors in Tuberculosis: Modulating Inflammation and Clinical Outcomes.” Front Immunol 8, 1948 (2017).
Bell et al., “The oligomeric structure of human granzyme A is a determinant of its extended substrate specificity.” Nat Struct Biol 10, 527-534 (2003).
Beresford et al., “Granzyme A loading induces rapid cytolysis and a novel form of DNA damage independently of caspase activation.” Immunity 10, 585-594 (1999).
Cohen et al., “Alveolar Macrophages Provide an Early Mycobacterium tuberculosis Niche and Initiate Dissemination.” Cell Host Microbe 24, 439-446 e434 (2018).
Chinnaraj et al., “Bioorthogonal Chemistry Enables Single-Molecule FRET Measurements of Catalytically Active Protein Disulfide Isomerase.” Chembiochem 22, 134-138 (2021).
Damiani et al., “Rab coupling protein associates with phagosomes and regulates recycling from the phagosomal compartment.” Traffic 5, 785-797 (2004).
Danwang et al., “Global epidemiology of venous thromboembolism in people with active tuberculosis: a systematic review and meta-analysis.” J Thromb Thrombolysis 51, 502-512 (2021).
Fan et al., “Tumor suppressor NM23-H1 is a granzyme A-activated DNase during CTL-mediated apoptosis, and the nucleosome assembly protein SET is its inhibitor.” Cell 112, 659-672 (2003).
Garzon-Tituana et al., “Granzyme A inhibition reduces inflammation and increases survival during abdominal sepsis.” Theranostics 11, 3781-3795 (2021).
Gaspar & Gibbins, “Thiol Isomerases Orchestrate Thrombosis and Hemostasis.” Antioxid Redox Signal 35, 1116-1133 (2021).
Hoft et al., “Bacille Calmette-Guerin vaccination enhances human γΔ T cell responsiveness to mycobacteria suggestive of a memory-like phenotype.” J Immunol 161, 1045-1054 (1998).
Hoft et al., “Trans-sialidase recombinant protein mixed with CpG motif-containing oligodeoxynucleotide induces protective mucosal and systemic Trypanosoma cruzi immunity involving CD8+ CTL and B cell-mediated cross-priming.” J Immunol 179:6889-6900 (2007).
Hmama et al., “Immunoevasion and immunosuppression of the macrophage by Mycobacterium tuberculosis.” Immunol Rev 264, 220-232 (2015).
Kaiserman et al., “The major human and mouse granzymes are structurally and functionally divergent.” J Cell Biol 175, 619-630 (2006).
Lindsay et al., “Rab coupling protein (RCP), a novel Rab4 and Rab11 effector protein.” J Biol Chem 277, 12190-12199 (2002).
Maitra et al., “Cell wall peptidoglycan in Mycobacterium tuberculosis: An Achilles' heel for the TB-causing pathogen.” FEMS Microbiol Rev 43, 548-575 (2019).
Martinvalet et al., “Granzyme A induces caspase-independent mitochondrial damage, a required first step for apoptosis.” Immunity 22, 355-370 (2005).
McClean & Tobin, “Macrophage form, function, and phenotype in mycobacterial infection: lessons from tuberculosis and other diseases.” Pathog Dis 74 (2016).
Metkar et al., “Human and mouse granzyme A induce a proinflammatory cytokine response.” Immunity 29, 720-733 (2008).
Nathan et al., “A philosophy of anti-infectives as a guide in the search for new drugs for tuberculosis.” Tuberculosis (Edinb) 88 Suppl 1, S25-33 (2008).
Napoli et al., “Increased granzyme levels in cytotoxic T lymphocytes are associated with disease severity in emergency department patients with severe sepsis.” Shock 37, 257-262 (2012).
Nardo et al., “Immortalization of Murine Bone Marrow-Derived Macrophages.” Methods Mol Biol 1784, 35-49 (2018).
Oliveira et al., “Protein disulfide isomerase plasma levels in healthy humans reveal proteomic signatures involved in contrasting endothelial phenotypes.” Redox Biol 22, 101142 (2019).
Park et al., “Mycobacterium tuberculosis Rv3463 induces mycobactericidal activity in macrophages by enhancing phagolysosomal fusion and exhibits therapeutic potential.” Sci Rep 9, 4246 (2019).
Rajaiah et al., “CD14 dependence of TLR4 endocytosis and TRIF signaling displays ligand specificity and is dissociable in endotoxin tolerance.” Proc Natl Acad Sci USA 112, 8391-8396 (2015).
Rasi et al., “Granzyme A Produced by γ9Δ2 T Cells Activates ER Stress Responses and ATP Production and Protects Against Intracellular Mycobacterial Replication Independent of Enzymatic Activity.” Front Immunol 12, 712678 (2021).
Rasi et al., “Improved Purification of Human Granzyme A/B and Granulysin Using a Mammalian Expression System.” Frontiers in Immunology 13 (2022).
Rossol et al., “LPS-induced cytokine production in human monocytes and macrophages.” Crit Rev Immunol 31, 379-446 (2011).
Stantchev et al., “Cell-type specific requirements for thiol/disulfide exchange during HIV-1 entry and infection.” Retrovirology 9, 97 (2012).
Spencer et al., “Only a subset of phosphoantigen-responsive γ9Δ2 T cells mediate protective tuberculosis immunity.” J Immunol 181, 4471-4484 (2008).
Spencer et al., “Granzyme A produced by γ(9)Δ(2) T cells induces human macrophages to inhibit growth of an intracellular pathogen.” PLoS Pathog 9, e1003119 (2013).
Taguchi & Mukai, “Innate immunity signalling and membrane trafficking.” Curr Opin Cell Biol 59, 1-7 (2019).
Tan et al., “Mechanisms of Toll-like Receptor 4 Endocytosis Reveal a Common Immune-Evasion Strategy Used by Pathogenic and Commensal Bacteria.” Immunity 43, 909-922 (2015).
Tsukamoto et al., “Lipopolysaccharide (LPS)-binding protein stimulates CD14-dependent Toll-like receptor 4 internalization and LPS-induced TBK1-IKK-IRF3 axis activation.” J Biol Chem 293, 10186-10201 (2018).
Uranga et al., “Granzyme A Is Expressed in Mouse Lungs during Mycobacterium tuberculosis Infection but Does Not Contribute to Protection In vivo.” PLoS One 11, e0153028 (2016).
Uranga-Murillo et al., “Biological relevance of Granzymes A and K during E. coli sepsis.” Theranostics 11, 9873-9883 (2021).
Wan et al., “Endothelial cell surface expression of protein disulfide isomerase activates beta1 and beta3 integrins and facilitates dengue virus infection.” J Cell Biochem 113, 1681-1691 (2012).
Wensink et al., “Granzymes A and K differentially potentiate LPS-induced cytokine response.” Cell Death Discov 2, 16084 (2016).
WHO, “Global tuberculosis report 2020.” Available from: world-wide-web at who.int/publications/i/item/9789240013131 2020 (2020).
WHO, “Global Tuberculosis Report.” Available from: world-wide-web at who.int/publications/i/item/9789240037021 2021 (2021).
Wilson et al., “Metabolic Programming of Macrophages: Implications in the Pathogenesis of Granulomatous Disease.” Front Immunol 10, 2265 (2019).
Worku & Hoft, “Differential effects of control and antigen-specific T cells on intracellular mycobacterial growth.” Infect Immun 71, 1763-1773 (2003).
Wu et al., “CD14: Biology and role in the pathogenesis of disease.” Cytokine Growth Factor Rev 48, 24-31 (2019).
Xia et al., “A Subset of Protective 79A2 T Cells Is Activated by Novel Mycobacterial Glycolipid Components.” Infect Immun 84, 2449-2462 (2016).
Young et al., “Therapeutic host-directed strategies to improve outcome in tuberculosis.” Mucosal Immunol 13, 190-204 (2020).
Zanoni et al., “CD14 controls the LPS-induced endocytosis of Toll-like receptor 4.” Cell 147, 868-880 (2011).

1. A method of treating or preventing infection of a human subject with Mycobacterium tuberculosis comprising providing to said subject an effective amount of a recombinant, homodimeric, enzymatically inactive mutant Granzyme A having a serine-*alanine substitution corresponding to position 212 of SEQ ID NO: 1.

2. The method of claim 1, wherein said subject has been diagnostically confirmed to have a Mycobacterium tuberculosis infection.

3. The method of claim 1, wherein said subject is at risk of contracting a Mycobacterium tuberculosis infection.

4. The method of claim 1, wherein recombinant, homodimeric, enzymatically inactive mutant Granzyme A has the sequence of SEQ ID NO: 4.

5. The method of claim 1, wherein recombinant, homodimeric, enzymatically inactive mutant Granzyme A is administered, such as intravenously, intramuscularly, subcutaneously, intranasal delivery, aerosol delivery, or inhaled.

6. The method of claim 1, wherein a nucleic acid (e.g., RNA) or expression construct, such as a viral or non-viral vector, that expresses recombinant, homodimeric, enzymatically inactive mutant Granzyme A is administered, such as intravenously, intramuscularly, subcutaneously, by intranasal delivery, by aerosol delivery, or inhaled.

7. The method of claim 1, wherein recombinant, homodimeric, enzymatically inactive mutant Granzyme A provided at about 50-200 nM.

8. The method of claim 1, wherein said method further comprises administering a second Mycobacterium tuberculosis therapy, such as an antibiotic.

9. The method of claim 1, wherein said Mycobacterium tuberculosis is drug resistant.

10. The method of claim 1, wherein recombinant, homodimeric, enzymatically inactive mutant Granzyme A, nucleic acid or expression construct is administered in an endotoxin free composition.

11. A recombinant, enzymatically inactive mutant Granzyme A having a serine-*alanine substitution corresponding to position 212 of SEQ ID NO: 1, or a nucleic acid (e.g., RNA or DAN), or an expression construct (e.g., viral or non-viral) encoding the same.

12. The mutant Granzyme A, nucleic acid or expression construct of claim 11, wherein said mutant Granzyme A has the sequence of SEQ ID NO: 2.

13. The mutant Granzyme A, nucleic acid or expression construct of claim 11, formulated for administration to a subject.

14. The mutant Granzyme A, nucleic acid or expression construct of claim 11, formulated as a unit dose form to deliver 50-200 nM mutant Granzyme A.

15. The mutant Granzyme A, nucleic acid or expression construct of claim 11, wherein said mutant Granzyme A is homodimeric.

16. The mutant Granzyme A, nucleic acid or expression construct of claim 11, wherein said mutant Granzyme A, nucleic acid or expression construct is lyophilized.

17. The mutant Granzyme A, nucleic acid or expression construct of claim 17, wherein said mutant Granzyme A, nucleic acid or expression construct is frozen.

18. A kit comprising the mutant Granzyme A, nucleic acid or expression construct of claim 11, disposed in a receptacle.

19. A method of producing the mutant Granzyme A of claim 11 comprising:
(a) transfecting a host cell with a nucleic acid encoding the mutant Granzyme A;
(b) culturing said host cell under conditions supporting expression of the mutant Granzyme A; and
(c) harvesting mutant Granzyme A from supernatants of said host cell.

20. The method of claim 19, further comprising purifying the mutant Granzyme A with column chromatography.
